(19) United States
(12) Patent Application Publication
(10) Pub. No.: US 2004/0229284 A1
(43) Pub. Date: Nov. 18, 2004

(54) Multiplex analysis of proteins

(75) Inventors: Paul A. Luciw, Davis, CA (US); Imran H. Khan, Davis, CA (US); Hsing-Jien Kung, Sacramento, CA (US)

Correspondence Address: QUINE INTELLECTUAL PROPERTY LAW GROUP, P.C., P O BOX 458, ALAMEDA, CA, 94501, US

(73) Assignee: The Regents of the University of California, Oakland, CA

(21) Appl. No.: 10/781,980

(22) Filed: Feb. 18, 2004

Related U.S. Application Data
(60) Provisional application No. 60/448,750, filed on Feb. 19, 2003.

(51) Int. Cl. C12Q 1/68; G01N 33/53; G01N 33/553

(52) U.S. Cl. 435/7.1; 436/526

(57) ABSTRACT

Several related methods for multiplexed detection of one or more posttranslational modifications of a plurality of proteins are provided. Methods for detecting one or more nucleic acid binding proteins are also provided. Use of such methods (e.g., methods of detecting phosphorylation of protein kinases) for diagnosis, prognosis and monitoring of disease is also included. Compositions, systems and kits that relate to each of the methods are described.

### CROSS REFERENCE TO RELATED APPLICATIONS

This application claims priority to and benefit of U.S. Provisional Patent Application No. 60/448,750, filed Feb. 19, 2003, “Multiplex Analysis of Proteins” by Paul A. Luciw et al., which is incorporated herein by reference in its entirety for all purposes.

### FIELD OF THE INVENTION

The present invention is in the field of protein detection. The invention includes methods of detecting posttranslational modification of proteins and methods of detecting nucleic acid binding proteins, and includes the use of such methods for diagnosis, prognosis and monitoring progression of disease.

### BACKGROUND OF THE INVENTION

Every cell expresses a large number of proteins, and protein expression patterns (which proteins are expressed and at what levels) vary, e.g., between cell types or depending on external stimuli or pathological state. Since many diseases are caused by, or correlated with, changes in protein expression patterns, comparing protein expression patterns between normal and disease conditions can reveal proteins whose altered expression is critical in causing the disease and can thus identify appropriate therapeutic targets. Similarly, protein expression profiles can be used in clinical diagnosis, prognosis and monitoring therapy. However, lack of large-scale protein screening methods is a major obstacle in profiling protein expression patterns. Techniques such as mass spectroscopy and two-dimensional electrophoresis can be used, but these methods are costly, time-consuming and not amenable to high throughput analysis. The basic ELISA (Enzyme-Linked Immunosorbent Assay) format can also be used to detect specific cellular proteins; however, this method is limited by the need for a relatively large sample size for detection of several cellular proteins in a test sample. New techniques for evaluating expression of several proteins simultaneously are thus desirable.

Similarly, new techniques for evaluating posttranslational modification of several proteins simultaneously are also desirable. Posttranslational modification of a protein can play an important role in its regulation. For example, posttranslational modification of a protein can control its enzymatic activity, its interaction with other molecules, its subcellular localization and/or its sensitivity to proteases. Aberrant modification of cellular proteins (e.g., aberrant glycosylation or phosphorylation) is a feature of many diseases. Mass spectroscopy or metabolic labeling of cells with radioisotopes can be used to detect posttranslational modifications, as can various immunodetection methods (e.g., immunoprecipitation, Western blotting, ELISAs). However, these methods tend to be applicable to only one or a few proteins at a time or require large sample sizes.

The present invention provides methods, compositions and related kits for detecting proteins and for detecting posttranslational modification of proteins that overcome the above noted difficulties. The methods, compositions and kits are simple and amenable to high throughput analysis, even with small sample sizes. A complete understanding of the invention will be obtained upon review of the following.

### SUMMARY OF THE INVENTION

The present invention provides several related strategies (including methods, kits and compositions) for detecting proteins and/or posttranslational modification of proteins. One class of embodiments provides particle-based methods for detecting posttranslational modification of a plurality of proteins. A related class of embodiments provides methods for diagnosis, prognosis and monitoring of disease using particle-based methods for detecting phosphorylation of a plurality of protein kinases. Another class of embodiments provides particle-based methods for detecting one or more nucleic acid binding proteins. Yet another class of embodiments provides array-based methods of detecting a plurality of posttranslational modifications of a plurality of proteins. Compositions, systems and kits that relate to each of the methods are also features of the invention.

In a first general class of methods, the invention provides particle-based methods of detecting the presence or absence of a first posttranslational modification of a plurality of proteins in a sample. (Related kits and compositions are also provided, as described in greater detail below.) In the methods, the sample comprising the proteins is provided, along with a single first detection reagent and a pooled population of a plurality of subsets of particles (e.g., microspheres). The particles in each subset comprise a capture reagent specific for at least one of the proteins (preferably, for one of the proteins), and the particles in each subset are distinguishable from those of every other subset. The first detection reagent provides an indication of the presence of the first posttranslational modification. The proteins are bound to the capture reagents and exposed to the first detection reagent. Then, each subset of particles is identified and the presence or absence of the first detection reagent on each subset of particles is detected, to determine whether each of the proteins comprises the first posttranslational modification.

The first posttranslational modification can be essentially any modification. For example, the first posttranslational modification can be phosphorylation, e.g., phosphorylation of a serine, threonine and/or tyrosine residue. Other examples of posttranslational modifications that can be detected include, but are not limited to, ubiquitination, sumoylation, glycosylation, prenylation, myristoylation, farnesylation, attachment of a fatty acid, attachment of a GPI anchor, acetylation, methylation and nucleotidylation (e.g., ADP-ribosylation).

A related general class of methods provides methods of diagnosing or monitoring disease by detecting the presence or absence of a phosphorylated amino acid residue (e.g., a phosphorylated serine, threonine, and/or tyrosine) in a plurality of protein kinases. (Again, related kits and compositions are also provided, as described in greater detail below.) In the methods, a sample comprising the protein kinases is provided, along with a single first detection reagent and a pooled population of a plurality of subsets of particles (e.g., microspheres). The particles in each subset comprise a capture reagent specific for at least one of the kinases (preferably, for one of the kinases), and the particles in each subset are distinguishable from those of every other subset. The first detection reagent provides an indication of the presence of the phosphorylated amino acid residue. The protein kinases are bound to the capture reagents and exposed to the first detection reagent. A kinase activity profile for the sample is generated by determining whether each of the kinases comprises the phosphorylated amino acid residue (and therefore whether each kinase is predicted to be active, partially active or inactive): each subset of particles is identified, and the presence or absence of the first detection reagent is detected on each subset of particles. The kinase activity profile for the sample is then compared with one or more control kinase activity profiles.

The control kinase activity profiles with which the profile of the sample is compared can comprise one or more of: a kinase activity profile for a normal, healthy cell; a kinase activity profile for a diseased cell; or a kinase activity profile for a second sample from the same source, taken at a different time. The method can be applied, for example, to neoplastic, infectious, neurological, inflammatory and cardiovascular disease, as well as other diseases in which differences are exhibited in the pattern of kinase phosphorylation compared to a normal healthy state. The kinase activity profile can indicate the type of disease present (e.g., type of cancer or infectious agent). Similarly, samples from a given individual taken at different times (e.g., before and after initiation of therapy) can be used to monitor response to therapy and/or to predict disease progression.

In another general class of embodiments, the invention provides methods of detecting the presence or absence of one or more nucleic acid binding proteins. In the methods, a sample comprising or suspected of comprising the one or more nucleic acid binding proteins is provided, along with one or more subsets of particles and one or more detection reagents. The particles in each subset comprise a nucleic acid binding site specific for at least one of the proteins (preferably, for one of the proteins), and the particles in each subset are distinguishable from those of every other subset. Each detection reagent provides an indication of the presence of at least one of the nucleic acid binding proteins. The one or more subsets of particles are exposed to the sample and then the one or more detection reagents are added to the exposed one or more subsets, or the one or more detection reagents are added to the sample and then the detection reagent(s) and the sample are exposed to the one or more subsets of particles. To determine whether each of the one or more proteins is present in the sample, each subset of particles is identified and the presence or absence of the one or more detection reagents is detected. Kits and compositions related to these methods are also features of the invention, as described in greater detail below.

Yet another general class of embodiments provides methods of detecting the presence or absence of a plurality of posttranslational modifications of a plurality of proteins in a sample. In the methods, the sample comprising the proteins is provided, along with a plurality of detection reagents and a solid support (e.g., a membrane, slide or plate) comprising a plurality of capture reagents. Each capture reagent is specific for at least one of the proteins (preferably, for one of the proteins), and each capture reagent is provided at a known, pre-determined position on the solid support. That is, the capture reagents form an array, such that each capture reagent (and thus, the protein bound to each capture reagent) can be identified by the position at which it is immobilized. Each detection reagent provides an indication of the presence of one of the posttranslational modifications. The proteins are bound to the capture reagents and exposed to the detection reagents. The presence or absence of each of the detection reagents is detected (at each position) to determine whether each of the proteins comprises each of the posttranslational modifications.

As discussed above, the present invention also includes compositions, e.g., for practicing the methods herein or which are produced by any of the methods. For example, the invention provides compositions comprising a single first detection reagent and a plurality of subsets of particles (e.g., microspheres). The particles in each subset comprise a capture reagent specific for at least one of a plurality of proteins comprising or suspected of comprising a first posttranslational modification, and the particles in each subset are distinguishable from those of every other subset. The first detection reagent provides an indication of the presence of the first posttranslational modification. Preferably, the particles in each subset comprise a capture reagent specific for one of the plurality of proteins.

The composition optionally also includes the plurality of proteins comprising or suspected of comprising the first posttranslational modification. Optionally, each of the plurality of proteins is associated with one of the subsets of particles (typically, via noncovalent association with the capture reagent). In one embodiment, the plurality of proteins comprises a plurality of protein kinases.

The first posttranslational modification can be essentially any modification. For example, the first posttranslational modification can be phosphorylation, e.g., phosphorylation of a serine, threonine and/or tyrosine residue. Other examples of posttranslational modifications include, but are not limited to, ubiquitination, sumoylation, glycosylation, prenylation, myristoylation, farnesylation, attachment of a fatty acid, attachment of a GPI anchor, acetylation, methylation and nucleotidylation (e.g., ADP-ribosylation).

In another aspect, the invention provides compositions that comprise one or more subsets of particles. The particles in each subset comprise a nucleic acid binding site specific for at least one nucleic acid binding protein (preferably, for one nucleic acid binding protein), and the particles in each subset are distinguishable from those of every other subset. The composition optionally also includes one or more nucleic acid binding proteins. Optionally, each nucleic acid binding protein is associated with one of the one or more subsets of particles.

In another aspect, the invention includes compositions that comprise a plurality of proteins comprising or suspected of comprising a plurality of posttranslational modifications, a solid support comprising a plurality of capture reagents, and a plurality of detection reagents. Each capture reagent is specific for at least one of the proteins (preferably, for one of the proteins), and each capture reagent is provided at a known, pre-determined position on the solid support. That is, the capture reagents form an array, such that each capture reagent (and thus, the protein bound to each capture reagent) can be identified by the position at which it is immobilized. Each detection reagent provides an indication of the presence of one of the posttranslational modifications.

Systems comprising each of the compositions provide an additional aspect of the invention, and kits are also features of the invention. For example, kits of the invention can include any of the compositions noted herein, instructions for practicing the methods herein, containers, packing materials and/or the like.

### BRIEF DESCRIPTION OF THE DRAWINGS

FIG. 1, Panels A-D schematically depict a multiplexed microsphere assay for phosphorylated proteins.

FIG. 2 is a bar graph illustrating multiplex detection of tyrosine phosphorylated proteins from sodium pervanadate activated Jurkat T-cells.

FIG. 3 illustrates immunoprecipitation and Western analysis of tyrosine phosphorylated proteins from sodium pervanadate activated Jurkat T-cells.

FIG. 4 is a line graph illustrating multiplex detection of tyrosine phosphorylated proteins from Jurkat T-cells activated by anti-CD3 antibody.

### DEFINITIONS

Unless defined otherwise, all technical and scientific terms used herein have the same meaning as commonly understood by one of ordinary skill in the art to which the invention pertains. The following definitions supplement those in the art and are directed to the current application and are not to be imputed to any related or unrelated case, e.g., to any commonly owned patent or application. Although any methods and materials similar or equivalent to those described herein can be used in the practice for testing of the present invention, the preferred materials and methods are described herein. Accordingly, the terminology used herein is for the purpose of describing particular embodiments only, and is not intended to be limiting.

As used in this specification and the appended claims, the singular forms “a,” “an” and “the” include plural referents unless the content clearly dictates otherwise. Thus, for example, reference to “a protein” includes a plurality of proteins, reference to “a cell” includes mixtures of cells, and the like.

As used herein, an “antibody” is a protein comprising one or more polypeptides substantially or partially encoded by immunoglobulin genes or fragments of immunoglobulin genes. The recognized immunoglobulin genes include the kappa, lambda, alpha, gamma, delta, epsilon and mu constant region genes, as well as myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively. A typical immunoglobulin (antibody) structural unit comprises a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (VL) and variable heavy chain (VH) refer to these light and heavy chains respectively. Antibodies exist as intact immunoglobulins or as a number of well-characterized fragments produced by digestion with various peptidases. Thus, for example, pepsin digests an antibody below the disulfide linkages in the hinge region to produce F(ab)′2, a dimer of Fab which itself is a light chain joined to VH-CH1 by a disulfide bond. The F(ab)′2 may be reduced under mild conditions to break the disulfide linkage in the hinge region thereby converting the (Fab′)₂ dimer into a Fab′ monomer. The Fab′ monomer is essentially a Fab with part of the hinge region (see, Fundamental Immunology, W. E. Paul, ed., Raven Press, N.Y. (1999), for a more detailed description of other antibody fragments). While various antibody fragments are defined in terms of the digestion of an intact antibody, one of skill will appreciate that such Fab′ fragments may be synthesized de novo either chemically or by utilizing recombinant DNA methodology. Thus, the term antibody, as used herein, includes antibodies or fragments either produced by the modification of whole antibodies or synthesized de novo using recombinant DNA methodologies. Antibodies include multiple or single chain antibodies, including single chain Fv (sFv or scFv) antibodies in which a variable heavy and a variable light chain are joined together (directly or through a peptide linker) to form a continuous polypeptide, and humanized or chimeric antibodies. Antibodies include polyclonal and monoclonal antibodies.

A “label” is a moiety that facilitates detection of a molecule. Common labels in the context of the present invention include fluorescent labels. Other labels include radionucleotides, enzymes, substrates, cofactors, inhibitors, colorimetric moieties, luminescent moieties, chemiluminescent moieties, magnetic particles, and the like. Patents teaching the use of such labels include U.S. Pat. Nos. 3,817,837; 3,850,752; 3,939,350; 3,996,345; 4,277,437; 4,275,149; and 4,366,241. Many labels are commercially available and can be used in the context of the invention.

A “microsphere” is a small spherical, or roughly spherical, particle. A microsphere typically has a diameter less than about 1000 micrometers (e.g., less than about 100 micrometers, optionally less than about ten micrometers). The microsphere can comprise any of a variety of materials (e.g., silica, polystyrene or another polymer) and can optionally have various surface chemistries (e.g., free carboxylic acid, amine, or hydrazide groups, among many others).

The term “nucleic acid” encompasses any physical string of monomer units that can be corresponded to a string of nucleotides, including a polymer of nucleotides (e.g., a typical DNA or RNA polymer), peptide nucleic acids, modified oligonucleotides (e.g., oligonucleotides comprising bases that are not typical to biological RNA or DNA in solution, such as 2′-O-methylated oligonucleotides), and the like. A nucleic acid can be e.g., single-stranded or double-stranded. Unless otherwise indicated, a particular nucleic acid sequence of this invention encompasses complementary sequences, in addition to the sequence explicitly indicated.

An “oligonucleotide” is a polymer comprising two or more nucleotides. The polymer can additionally comprise non-nucleotide elements such as labels, quenchers, blocking groups, and/or the like. The nucleotides of the oligonucleotide can be deoxyribonucleotides, ribonucleotides or nucleotide analogs, can be natural or non-natural, and can be unsubstituted, unmodified, substituted or modified. The nucleotides can be linked by phosphodiester bonds, or by phosphorothioate linkages, methylphosphonate linkages, boranophosphate linkages, and/or the like. A “synthetic oligonucleotide” or a “chemically synthesized oligonucleotide” is an oligonucleotide made through in vitro chemical synthesis, as opposed to an oligonucleotide made either in vitro or in vivo by a template-directed, enzyme-dependent reaction.

A “polypeptide” is a polymer comprising two or more amino acid residues (e.g., a peptide or a protein). The polymer can additionally comprise non-amino acid elements such as labels, quenchers, blocking groups, and/or the like and can optionally comprise modifications such as glycosylation and/or the like. The amino acid residues of the polypeptide can be natural or non-natural and can be unsubstituted, unmodified, substituted or modified. A “synthetic peptide” or a “chemically synthesized peptide” is a polypeptide made through in vitro chemical synthesis, as opposed to a polypeptide made either in vitro or in vivo by a template-directed, enzyme-dependent reaction.

A “posttranslational modification” of a protein is an enzymatic transformation that occurs following translation of some or all of the protein's amino acid residues. Typically, posttranslational modification involves attachment of a small chemical group (or groups) to a functional group of certain amino acid residues (e.g., the epsilon amino group of lysine) or to the protein's terminal amino or carboxyl group. Examples include, but are not limited to, phosphorylation, glycosylation, acetylation, lipidation (e.g., prenylation, farnesylation, myristoylation, attachment of a fatty acid or a GPI anchor), ubiquitination, sumoylation, hydroxylation, methylation and nucleotidylation (e.g., ADP-ribosylation).

The term “recombinant” indicates that the material (e.g., a nucleic acid or a protein) has been artificially or synthetically (non-naturally) altered by human intervention. The alteration can be performed on the material within, or removed from, its natural environment or state. For example, a “recombinant nucleic acid” is one that is made by recombining nucleic acids, e.g., during cloning, DNA shuffling or other procedures, while a “recombinant polypeptide” or “recombinant protein” is a polypeptide or protein which is produced by expression of a recombinant nucleic acid.

A capture reagent “specific for” a protein in a mixture of proteins has a higher affinity for that protein than for any other protein in the mixture. Typically, the capture reagent binds the protein for which it is specific at least about 10 times more tightly (and preferably at least about 100 times more tightly, at least about 1000 times more tightly, or even at least about 10,000 times more tightly) than any other protein in the mixture, e.g., under typical assay conditions. Similarly, a nucleic acid binding site “specific for” a nucleic acid binding protein in a mixture of nucleic acid binding proteins has a higher affinity for that protein than for any other nucleic acid binding protein in the mixture. Typically, the nucleic acid binding site binds the nucleic acid binding protein for which it is specific at least about 10 times more tightly (and preferably at least about 100 times more tightly, at least about 1000 times more tightly, or even at least about 10,000 times more tightly) than any other protein in the mixture, e.g., under typical assay conditions.

A variety of additional terms are defined or otherwise characterized herein.

### DETAILED DESCRIPTION

The present invention provides several related methods that provide for efficient multiplexed detection of proteins and/or posttranslational modification of proteins. Compositions, systems and kits that relate to each of the methods are also features of the invention.

Detection of Posttranslational Modification: Particle Assay

One aspect of the present invention provides new methods and related compositions and kits for rapid, efficient and quantitative detection of posttranslational modification of multiple proteins in a single reaction. Thus, in one aspect, the invention includes methods of detecting the presence or absence of a first posttranslational modification of a plurality of proteins in a sample. In the methods, the sample comprising the proteins is provided, along with a single first detection reagent and a pooled population of a plurality of subsets of particles. The particles in each subset comprise a capture reagent specific for at least one of the proteins (preferably, for one of the proteins), and the particles in each subset are distinguishable from those of every other subset. The first detection reagent provides an indication of the presence of the first posttranslational modification. The proteins are bound to the capture reagents and exposed to the first detection reagent. Then, each subset of particles is identified and the presence or absence of the first detection reagent on each subset of particles is detected, to determine whether each of the proteins comprises the first posttranslational modification.

The first posttranslational modification can be essentially any modification. For example, the first posttranslational modification can be phosphorylation, e.g., phosphorylation of a serine, threonine and/or tyrosine residue. Other examples of posttranslational modifications that can be detected include, but are not limited to, ubiquitination, sumoylation, glycosylation, prenylation, myristoylation, farnesylation, attachment of a fatty acid, attachment of a GPI anchor, acetylation, methylation and nucleotidylation (e.g., ADP-ribosylation).

Binding of the proteins to the capture reagents and exposure of the proteins to the first detection reagent can occur simultaneously or sequentially, in various orders. For example, in one embodiment, the pooled population of subsets of particles is exposed to the sample, and the first detection reagent is added to the exposed pooled population. In this embodiment, the exposed pooled population is optionally washed prior to the addition of the first detection reagent (e.g., with a solution comprising a buffer, salts, detergent and/or a blocking agent, or the like). As another example, in another embodiment the sample and the first detection reagent are combined and then are combined with the pooled population of subsets of particles. The particles can optionally be washed prior to detection of the first detection reagent. The wash(es) can be included, e.g., for increased sensitivity and/or specificity, or omitted, e.g., for speed and simplicity.

In one class of embodiments, the particles are microspheres. In preferred embodiments, the particles are microspheres, and the microspheres of each subset are distinguishable from those of the other subsets on the basis of their fluorescent emission spectra and/or their diameter (i.e., their size).

The capture reagent for a particular protein can be essentially any molecule that binds specifically to that protein. For example, a capture reagent can comprise a nucleic acid (e.g., an oligonucleotide, a nucleic acid binding site, an aptamer), a polypeptide (e.g., an antibody, a recombinant protein, a synthetic peptide), a substrate analog (e.g., a molecule that is a structural analog of an enzyme's substrate but that reacts very slowly or not at all and thus inhibits the enzyme by occupying its active site) and/or a small molecule (e.g., a ligand). A single subset of particles typically (but not necessarily) comprises a single type of capture reagent, while different subsets can comprise the same or different types of capture reagents. For example, one subset can comprise an antibody specific for a first protein while a second subset comprises an antibody specific for a second protein, or one subset can comprise an antibody specific for a first protein while a second subset comprises a single-stranded or double-stranded oligonucleotide binding site for a second protein. The capture reagent can be covalently or noncovalently associated with the particles, as described in greater detail in the “Microspheres” section below. For example, the capture reagent can be covalently coupled to carboxylate-modified particles via a carbodiimide coupling method or to maleimide-modified particles via a thiol-maleimide interaction. As another example, a biotinylated capture reagent can be noncovalently associated with streptavidin-modified particles, or a GST-tagged or polyhistidine-tagged recombinant protein can be noncovalently associated with glutathione or Ni²⁺ coated particles.

Similarly, the first detection reagent can be essentially any molecule capable of specifically recognizing the first posttranslational modification. For example, the first detection reagent can comprise a nucleic acid (e.g., an oligonucleotide, an aptamer), a polypeptide (e.g., an antibody, a synthetic peptide, a recombinant protein, e.g., a recombinant protein comprising an SH2, PTB, 14-3-3, FHA, WD40 and/or WW domain capable of binding a phosphorylated residue or peptide), and/or a small molecule. In one class of embodiments, the first detection reagent is an antibody specific for a phosphorylated tyrosine, serine and/or threonine residue (e.g., a monoclonal antibody against phosphoserine, phosphothreonine or phosphotyrosine, a polyclonal antibody against phosphothreonine and phosphoserine, or a polyclonal antibody against phosphotyrosine, among many other possible examples). In other embodiments, the first detection reagent is an antibody specific for another posttranslational modification; for example, an antibody specific for ubiquitin, a SUMO polypeptide, a carbohydrate moiety, an acetyl group, a prenyl group, or the like. In other embodiments, the first detection reagent is a lectin.

The first detection reagent can be labeled and detected directly, or it can be indirectly detected. Thus, in one class of embodiments, the first detection reagent comprises a first fluorescent label. In this class of embodiments, detecting the presence or absence of the first detection reagent comprises detecting a first fluorescent signal from the first label. In other embodiments, the first detection reagent is not fluorescently labeled, but is instead detected by adding a labeled secondary agent that binds the first detection reagent and detecting a signal from the labeled secondary agent. For example, the first detection reagent can be biotinylated, and the secondary agent can be fluorescently labeled streptavidin. Fluorescent emission by the first label (whether on the first detection reagent or on the secondary agent) is typically distinguishable from any fluorescent emission by the particles. For example, if orange and red emitting microspheres (e.g., from Luminex Corp.) are used as the particles, Alexa Fluor 488 or R-phycoerythrin can be used as the label on the first detection reagent or the secondary agent. As another example, if green emitting microspheres (e.g., from Beckman Coulter) are used as the particles, PC5 can be used for the label. Many suitable fluorescent label-fluorescent particle combinations are possible, and selection of an appropriate combination for a particular application is routine for one of skill. Details regarding labels and detection strategies can be found, e.g., in The Handbook of Fluorescent Probes and Research Products, Ninth Edition by Richard P. Haughland, published by Molecular Probes, Inc. The Handbook is available in print from Molecular Probes, or on-line on the world wide web at www.molecularprobes.com. Fluorescent emission by the label can be conveniently detected, and subsets of particles can be identified, using, e.g., a flow cytometer or similar instrument.

The methods can be qualitative or quantitative. For example, the first fluorescent signal from a first detection reagent comprising a first fluorescent label can be detected to indicate the presence or absence of the first detection reagent and therefore of the first posttranslational modification, or the first fluorescent signal can be quantitated to provide an indication of the extent of modification. For example, microspheres that have captured proteins from a sample can be analyzed in parallel with control microsphere sets (e.g., microspheres exposed to known amounts of a control protein whose modification status is known, e.g., a recombinant protein). One of skill can determine appropriate conditions for a quantitative assay by methods known in the art (e.g., using limiting concentrations of the proteins in the sample and non-limiting concentrations of capture and detection reagents, appropriate controls, and the like).

The proteins to be analyzed can be essentially any desired proteins. For example, the proteins can comprise an endogenous cellular protein or proteins (e.g., an intracellular protein, a plasma membrane protein and/or a secreted protein encoded by the cell's nuclear, mitochondrial and/or chloroplast genome) and/or a protein or proteins encoded by an infectious agent (e.g., a pathogenic virus, bacterium, protist, fungus or the like). In one embodiment, the plurality of proteins comprises a plurality of protein kinases.

Similarly, the sample comprising the proteins can be obtained or prepared from essentially any desired source. For example, the sample can be derived from an animal (e.g., a mammal, an invertebrate or an insect), a human, a plant, a cultured cell, and/or a microorganism. The sample can be derived, e.g., from a tissue, a biopsy or a tumor, e.g., from a human patient. The sample can comprise, for example, one or more of: a cell lysate (e.g., a lysate of cultured cells, a tissue lysate or a lysate of peripheral blood cells), an intercellular fluid, a conditioned culture medium or a bodily fluid (e.g., blood, serum, saliva, urine, sputum or spinal fluid).

The method can comprise additional steps. For example, at least one of the subsets of particles can be recovered, e.g., for additional analysis of the protein(s) associated with those particles. The particles can be recovered, for example, by being sorted into a separate sample tube by a flow cytometer and recovered by centrifugation (or by magnetic attraction if the particles are paramagnetic). The additional analysis can verify that the method is performing as expected. For example, a subset of particles can be recovered and analyzed, e.g., by mass spectroscopy, to determine if the intended protein and substantially only the intended protein was captured by the capture reagent. Alternatively, the additional analysis can provide new information. For example, the sample can be prepared and analyzed under mild conditions such that noncovalently associated protein complexes are not disrupted. In this example, a subset of particles can be recovered and analyzed, e.g., by mass spectroscopy or immunoassay, to determine what, if any, other proteins are associated with the protein captured by the capture reagent.

The method can be extended, providing further multiplexing capability with the addition of a second (and optional third, fourth, etc.) detection reagent. For example, a second detection reagent can be provided. The proteins are exposed to the second detection reagent (typically at the same time they are exposed to the first detection reagent), and the presence or absence of the second detection reagent on each subset of particles is detected (typically at the same time the first detection reagent is detected). The second detection reagent can provide an indication of the presence of a second posttranslational modification. To list only a few of the possible examples, the first detection reagent can be specific for tyrosine phosphorylation and the second for serine phosphorylation, the first detection reagent can be specific for phosphorylation (tyrosine, threonine and/or serine) and the second for ubiquitination, or the first detection reagent can be specific for glycosylation and the second for ubiquitination. Alternatively, the second detection reagent can provide an indication of the presence of a specific protein (e.g., the second detection reagent can be an antibody to a protein that forms a complex with one of the proteins captured by the capture reagents), protein family, or the like. Like the first detection reagent, the second detection reagent can itself be labeled, or it can be indirectly detected by use of a secondary agent. The label for the second detection reagent is typically distinguishable from that for the first detection reagent (and from the particles, if applicable). For example, if red and orange fluorescent beads (e.g., from Luminex Corp.) are used as the particles, one detection reagent can be labeled with Alexa Fluor 488 (Molecular Probes, Inc.) and the other detection reagent can be labeled with R-phycoerythrin.

The methods can be used, for example, for diagnosis, prognosis and/or monitoring of disease. The modification status of the plurality of proteins in the sample (determined by detecting the presence or absence of the first detection reagent on each subset of particles) can, e.g., be compared to the modification status of the proteins in one or more control samples (e.g., samples from a normal, healthy individual, from a diseased individual, or from the same individual but taken at a different time). For example, the modification status of the proteins can indicate the type of disease present in an individual, or samples from a given individual taken at different times (e.g., before and after initiation of therapy) can be used to monitor response to therapy and/or to predict disease progression.

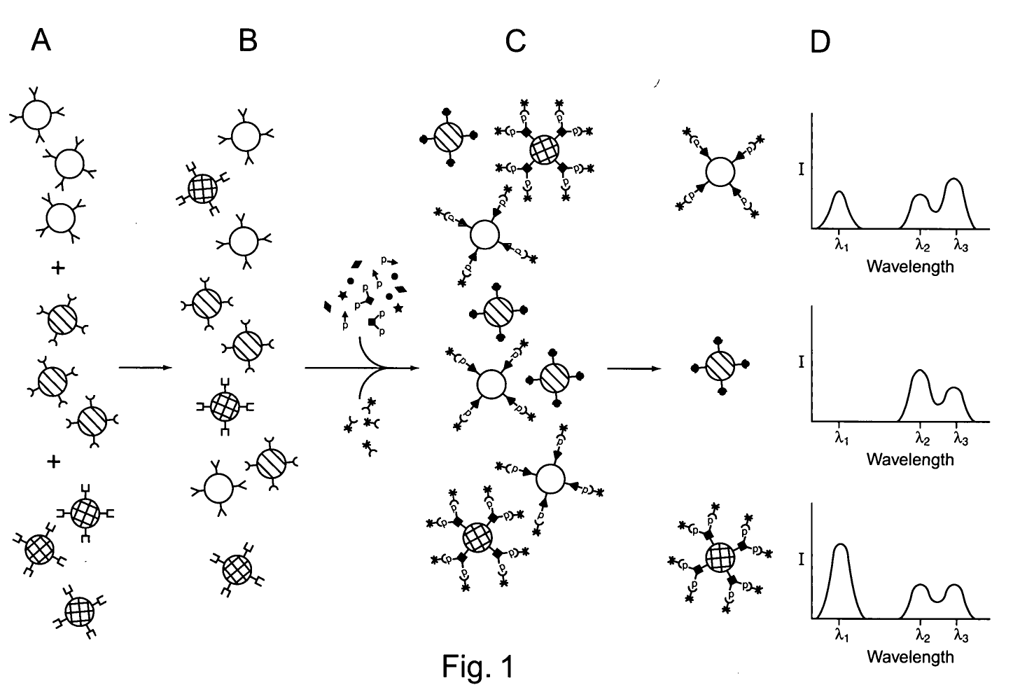

The basic method is schematically illustrated in FIG. 1. In this figure, three subsets of uniquely labeled microspheres (open and hatched circles) are each coated with a specific capture reagent (Panel A). The subsets are mixed into one reaction container (Panel B), and the sample is added. Proteins in the sample (represented by various solid shapes) are captured by the appropriate capture reagents (represented by complementary shapes). The detection reagent (in this example, a labeled detection reagent against phosphotyrosine, with the label represented by an asterisk) is also added. The detection reagent binds to phosphorylated tyrosine residues in the proteins (Panel C). The mixture is then analyzed in a flow cytometer or other instrument designed to identify each microsphere species (and therefore the captured protein, since each microsphere subset is uniquely labeled and coated with a unique capture reagent) and to measure the detection reagent (Panel D). In this example, the microsphere subsets are distinguishable by their differing fluorescent emission spectra (schematically illustrated in Panel D by the differing intensities (I) of emission at λ₂ and λ₃). Emission by the detection reagent (schematically illustrated at λ₁) is distinguishable from emission by the microspheres. As noted previously, the method is optionally quantitative, since the intensity of emission by the detection reagent is proportional to the amount of detection reagent bound to a captured protein (and therefore, under appropriate conditions, to the amount of that protein initially present in the sample).

As mentioned previously and discussed in greater detail below, compositions and kits related to the methods are also features of the invention. For example, compositions comprising a single first detection reagent and a plurality of subsets of particles (e.g., microspheres) are provided. The particles in each subset comprise a capture reagent specific for at least one of a plurality of proteins comprising or suspected of comprising a first posttranslational modification, and the particles in each subset are distinguishable from those of every other subset. The first detection reagent provides an indication of the presence of the first posttranslational modification. Optionally, the composition also includes the plurality of proteins comprising or suspected of comprising the first posttranslational modification; each protein is optionally associated with one of the subsets of particles (typically, via noncovalent association with the capture reagent). As another example, kits, e.g., kits facilitating practice of the invention, are provided. For example, a kit comprising each of the components of the composition and instructions for using the composition to detect at least one posttranslational modification, packaged in one or more containers, is a feature of the invention. Optionally, the kit includes instructions for diagnosis, prognosis and/or monitoring of disease by detecting the presence or absence of the posttranslational modification(s).

Uses and Advantages

The methods, kits and compositions enable rapid, efficient and quantitative analysis of multiple proteins in a single reaction. An important feature is the use of a single first detection reagent to detect the presence or absence of the first posttranslational modification on all the proteins in the reaction (as opposed to the use of a plurality of first detection reagents, each of which detects the first posttranslational modification of only one of the proteins, for example).

It will be evident to one of skill that, for a protein that is posttranslationally modified, detecting the presence or absence of the posttranslational modification for that protein is synonymous with detecting the presence or absence of the modified protein itself.

The methods, kits and compositions have a number of potential uses. They can be used in basic research, to analyze posttranslational modification of essentially any proteins. They can also be used in basic biomedical research, to investigate molecular mechanisms of disease in all phyla, and in clinical practice, for disease diagnosis, for disease prognosis, and for monitoring host responses to therapeutic regimens (in all phyla). That is, information on the presence and posttranslational modification (and therefore the predicted activity) of proteins can be used to diagnose a variety of diseases, to predict disease progression and to monitor response to therapies. These kits, compositions and methods apply to a wide variety of diseases, including neoplastic, infectious, neurological, inflammatory and cardiovascular diseases, as well as other diseases in which differences are exhibited in the pattern of protein expression and/or modification compared to the normal healthy state.

For example, the sample can be a lysate from a tissue biopsy from an individual having or suspected of having a disease. Comparisons can be made between the biopsy test sample and controls representing normal healthy biopsy material and/or known disease samples. For example, protein kinases, nuclear hormone receptors and mediators of apoptosis can be analyzed in tumor tissue. Similarly, the multiplex detection method can be used to detect specific proteins that are markers for disease in bodily fluids (e.g., serum, saliva, or urine). For prognosis and/or monitoring response to therapy, samples for a given individual can be taken and analyzed at different time points, to measure and compare changes in expression and/or posttranslational modification (and therefore predicted activity) of several specific cellular proteins.

Changes in the phosphorylation state of various proteins have been correlated with a number of diseases. Phosphorylation of protein kinases provides one example of how posttranslational modification can be analyzed to provide information about disease states. The activation state of many protein kinases is controlled by phosphorylation of the protein kinases. Thus, the activation state of various protein kinases can be analyzed by determining their phosphorylation status. Aberrant activation or inactivation of kinases has been implicated in a number of diseases (see, e.g., Johnson and Lapadat (2002) Science 298:1911-1912; Manning et al. (2002) Science 298: 1912-1934; Normanno et al. (2003) “Epidermal growth factor receptor tyrosine kinase inhibitors (EGFR-TKIs): simple drugs with a complex mechanism of action?” J Cell Physiol 194:13-9; Deininger et al. (2000) “The molecular biology of chronic myeloid leukemia” Blood 96:3343-56; and Sawyers (2002) “Rational therapeutic intervention in cancer: kinases as drug targets” Curr Opin Genet Dev 12:111-5). Which kinases are aberrantly active or inactive, for example, can indicate the type of tumor. Changes in kinase phosphorylation (and therefore activity) over time can be used to monitor response to therapy or to predict disease progression. As one example, anti-kinase antibodies can be attached to microspheres to capture specific tyrosine kinases in a tumor cell lysate. Incubation with an anti-phosphotyrosine antibody can label proteins with phosphorylated tyrosines. Flow cytometry or the like can be used to detect microspheres that are labeled with the anti-phosphotyrosine antibody, thus indicating which kinases are phosphorylated and thus which kinases are active, partially active or inactive. This provides the phosphotyrosine activity profile of the tumor for diagnosis, prognosis and therapeutic monitoring.

As another example, aberrant glycosylation has been implicated in a number of diseases. See, e.g., Muntoni et al (2002) “Defective glycosylation in muscular dystrophy” Lancet 360:1419-21; Freeze (2002) “Human disorders in N-glycosylation and animal models” Biochim Biophys Acta. 1573:388-93; Jaeken and Matthijs (2001) “Congenital disorders of glycosylation” Annu Rev Genomics Hum Genet. 2:129-51; Shane and Hart (1999) “Dynamic cytoskeletal glycosylation and neurodegenerative disease” Trends Glycosci Glycotech 11:355-370; Chui et al. (2001) “Genetic remodeling of protein glycosylation in vivo induces autoimmune disease” Proc. Nat. Acad. Sci. 98:1142-1147; and Isenberg and Rademacher (eds.) Abnormalities of IgG glycosylation and immunological disorders (1996) Jossey-Bass.

As another example, proteins encoded by infectious agents associated with disease can be detected for the purposes of identifying the agent. Expression and/or modification of selected proteins expressed by the infected host can also be detected for diagnostic and prognostic purposes.

As described above, the process can be modified (e.g., by preparing a tissue cell lysate under mild disruption conditions) to detect protein complexes and to subsequently determine the composition of these protein complexes. This information can also be applicable to disease diagnosis, prognosis and therapeutic monitoring. See, e.g., Wobus et al. (2002) “CD44 associates with EGFR and erbB2 in metastasizing mammary carcinoma cells” Appi Immunohistochem Mol Morphol 10:34-9; Koutsami et al. (2002) “Prognostic factors in non-small cell lung carcinoma” Anticancer Res 22(1A):347-74; and de Jong et al. (1997) “BCR/ABL-induced leukemogenesis causes phosphorylation of Hef1 and its association with Crkl” J Biol Chem 272:32649-55.

A first advantage of the methods, kits, and compositions is the small size of test sample that is required. A second advantage is the ability to detect the presence, activity and/or posttranslational modification of numerous target molecules (e.g., disease associated proteins) simultaneously in one reaction container. A third advantage is the rapidity of determining the profile (presence, activity and/or modification of numerous proteins in numerous biochemical pathways, e.g., for disease diagnosis and prognosis, e.g., for analysis of multiple cell signaling pathways in cancer). A fourth advantage is the ability to quantitate reactive proteins in the test sample. A fifth advantage is the ability to directly compare protein profiles of normal, healthy and disease-associated proteins. A sixth advantage is that specific proteins can be recovered and subjected to additional analysis (e.g., mass spectroscopy). A seventh advantage is the ability to simultaneously detect several different protein modifications in the test sample (e.g., phosphorylation and ubiquitination). An eighth advantage is the ability to simultaneously detect proteins that bind specific capture proteins and proteins that bind specific nucleic acid sequences, in one reaction container.

Many of these features and advantages are shared by the other aspects of the invention.

Disease Diagnosis, Prognosis and Monitoring by Detection of Phosphorylation of Protein Kinases

As mentioned previously, determining the phosphorylation state, and thus the activation state, of various protein kinases can be useful in disease diagnosis and prognosis and in monitoring response to therapy. Thus, one aspect of the invention provides methods of and related kits and compositions for diagnosing or monitoring disease by detecting the presence or absence of a phosphorylated amino acid residue in a plurality of protein kinases. In the methods, a sample comprising the protein kinases is provided, along with a single first detection reagent and a pooled population of a plurality of subsets of particles. The particles in each subset comprise a capture reagent specific for at least one of the kinases (preferably, for one of the kinases), and the particles in each subset are distinguishable from those of every other subset. The first detection reagent provides an indication of the presence of the phosphorylated amino acid residue. The protein kinases are bound to the capture reagents and exposed to the first detection reagent. A kinase activity profile for the sample is generated by determining whether each of the kinases comprises the phosphorylated amino acid residue (and therefore whether each kinase is predicted to be active, partially active or inactive): each subset of particles is identified, and the presence or absence of the first detection reagent is detected on each subset of particles. The kinase activity profile for the sample is then compared with one or more control kinase activity profiles.

Binding of the protein kinases to the capture reagents and exposure of the protein kinases to the first detection reagent can occur simultaneously or sequentially, in various orders. For example, in one embodiment, the pooled population of subsets of particles is exposed to the sample, and the first detection reagent is added to the exposed pooled population. In this embodiment, the exposed pooled population is optionally washed prior to the addition of the first detection reagent (e.g., with a solution comprising a buffer, salts, detergent and/or a blocking agent, or the like). As another example, in another embodiment the sample and the first detection reagent are combined and then are combined with the pooled population of subsets of particles. The particles can optionally be washed prior to detection of the first detection reagent. The wash(es) can be included, e.g., for increased sensitivity and/or specificity, or omitted, e.g., for speed and simplicity.

The phosphorylated amino acid residue can be a serine, threonine and/or tyrosine residue (that is, phosphorylated serines, phosphorylated threonines, or phosphorylated tyrosines, or any combination thereof, can be detected).

In one class of embodiments, the particles are microspheres. In preferred embodiments, the particles are microspheres, and the microspheres of each subset are distinguishable from those of the other subsets on the basis of their fluorescent emission spectra and/or their diameter (i.e., their size).

The capture reagent for a particular kinase can be essentially any molecule that binds specifically to that kinase. For example, a capture reagent can comprise a nucleic acid (e.g., an oligonucleotide, a nucleic acid binding site, an aptamer), a polypeptide (e.g., an antibody, a recombinant protein, a synthetic peptide), a substrate analog (e.g., a molecule that is a structural analog of an enzyme's substrate but that reacts very slowly or not at all and thus inhibits the enzyme by occupying its active site) and/or a small molecule (e.g., a ligand). In one class of embodiments, the capture reagents are antibodies. A single subset of particles typically (but not necessarily) comprises a single type of capture reagent, while different subsets can comprise the same or different types of capture reagents. For example, one subset can comprise an antibody specific for a first kinase while a second subset comprises an antibody specific for a second kinase, or one subset can comprise an antibody specific for a first kinase while a second subset comprises a recombinant protein that binds the second kinase. The capture reagent can be covalently or noncovalently associated with the particles, as noted for the embodiments above.

Similarly, the first detection reagent can be essentially any molecule capable of specifically recognizing a phosphorylated amino acid residue or peptide. For example, the first detection reagent can comprise a nucleic acid (e.g., an oligonucleotide, an aptamer), a polypeptide (e.g., an antibody, a synthetic peptide, a recombinant protein, e.g., a recombinant protein comprising an SH2, PTB, 14-3-3, FHA, WD40 and/or WW domain capable of binding a phosphorylated residue or peptide) and/or a small molecule. In one class of embodiments, the first detection reagent is an antibody specific for a phosphorylated tyrosine, serine and/or threonine residue (e.g., a monoclonal antibody against phosphoserine, phosphothreonine or phosphotyrosine, a polyclonal antibody against phosphothreonine and phosphoserine, or a polyclonal antibody against phosphotyrosine, among many other possible examples).

The first detection reagent can be labeled and detected directly, or it can be indirectly detected. Thus, in one class of embodiments, the first detection reagent comprises a first fluorescent label. In this class of embodiments, detecting the presence or absence of the first detection reagent comprises detecting a first fluorescent signal from the first label. In other embodiments, the first detection reagent is not fluorescently labeled, but is instead detected by adding a labeled secondary agent that binds the first detection reagent and detecting a signal from the labeled secondary agent. For example, the first detection reagent can be biotinylated, and the secondary agent can be fluorescently labeled streptavidin. As noted above, fluorescent emission by the first label (whether on the first detection reagent or on the secondary agent) is typically distinguishable from any fluorescent emission by the particles, and many suitable fluorescent label-fluorescent particle combinations are possible. Fluorescent emission by the label can be conveniently detected, and subsets of particles can be identified, using, e.g., a flow cytometer or similar instrument.

The methods can be qualitative or quantitative. For example, the fluorescent signal from a first detection reagent comprising a fluorescent label can be detected to indicate the presence or absence of the first detection reagent and therefore of the phosphorylated amino acid residue, or the fluorescent signal can be quantitated to provide an indication of the extent of phosphorylation. For example, microspheres that have captured protein kinases from a sample can be analyzed in parallel with control microsphere sets (e.g., microspheres exposed to known amounts of a control protein kinase whose phosphorylation status is known). One of skill can determine appropriate conditions for a quantitative assay by methods known in the art (e.g., using limiting concentrations of the proteins in the sample and non-limiting concentrations of capture and detection reagents, appropriate controls, and the like).

The kinases to be analyzed can be essentially any desired kinases. For example, the kinases can comprise an endogenous cellular protein or proteins (e.g., an intracellular protein, a plasma membrane protein and/or a secreted protein encoded by the cell's nuclear, mitochondrial and/or chloroplast genome) and/or a protein or proteins encoded by an infectious agent (e.g., a pathogenic virus, bacterium, protist, fungus or the like).

Similarly, the sample comprising the kinases can be obtained or prepared from essentially any desired source. For example, the sample can be derived from an animal (e.g., a mammal, an invertebrate or an insect), a human, a plant, a cultured cell, and/or a microorganism. The sample can be derived, e.g., from a tissue, a biopsy or a tumor, e.g., from a human patient. The sample can comprise, for example, one or more of: a cell lysate (e.g., a lysate of cultured cells, a tissue lysate or a lysate of peripheral blood cells), an intercellular fluid, a conditioned culture medium or a bodily fluid (e.g., blood, serum, saliva, urine, sputum or spinal fluid).

The control kinase activity profiles with which the profile of the sample is compared can comprise one or more of: a kinase activity profile for a normal, healthy cell; a kinase activity profile for a diseased cell; or a kinase activity profile for a second sample from the same source, taken at a different time. The method can be applied, for example, to neoplastic, infectious, neurological, inflammatory and cardiovascular disease, as well as other diseases in which differences are exhibited in the pattern of kinase phosphorylation compared to a normal healthy state. The kinase activity profile can indicate the type of disease present (e.g., type of cancer or infectious agent). Similarly, samples from a given individual taken at different times (e.g., before and after initiation of therapy) can be used to monitor response to therapy and/or to predict disease progression.

The method can comprise additional steps. For example, at least one of the subsets of particles can be recovered, e.g., for additional analysis of the kinase and/or any other protein(s) associated with those particles. The particles can be recovered, for example, by being sorted into a separate sample tube by a flow cytometer and recovered by centrifugation (or by magnetic attraction if the particles are paramagnetic). The additional analysis can verify that the method is performing as expected. For example, a subset of particles can be recovered and analyzed, e.g., by mass spectroscopy, to determine if the intended kinase and substantially only the intended kinase was captured by the capture reagent. Alternatively, the additional analysis can provide new information. For example, the sample can be prepared and analyzed under mild conditions such that noncovalently associated protein complexes are not disrupted. In this example, a subset of particles can be recovered and analyzed, e.g., by mass spectroscopy or immunoassay, to determine what, if any, other proteins are associated with the kinase captured by the capture reagent.

The method can be extended, providing further multiplexing capability with the addition of a second (and optional third, fourth, etc.) detection reagent. For example, a second detection reagent can be provided. The protein kinases are exposed to the second detection reagent (typically at the same time they are exposed to the first detection reagent), and the presence or absence of the second detection reagent on each subset of particles is detected (typically at the same time the first detection reagent is detected). The second detection reagent can provide an indication of the presence of a second posttranslational modification. To list only a few of the possible examples, the first detection reagent can be specific for tyrosine phosphorylation and the second for serine phosphorylation, the first detection reagent can be specific for phosphorylation (tyrosine, threonine and/or serine) and the second for ubiquitination, or the first detection reagent can be specific for serine phosphorylation and the second for ubiquitination. Alternatively, the second detection reagent can provide an indication of the presence of a specific protein (e.g., the second detection reagent can be an antibody to a protein that forms a complex with one of the proteins captured by the capture reagents), protein family, or the like. Like the first detection reagent, the second detection reagent can itself be labeled, or it can be indirectly detected by use of a secondary agent. The label for the second detection reagent is typically distinguishable from that for the first detection reagent (and from the particles, if applicable). For example, if red and orange fluorescent beads (e.g., from Luminex Corp.) are used as the particles, one detection reagent can be labeled with Alexa Fluor 488 (Molecular Probes, Inc.) and the other detection reagent can be labeled with R-phycoerythrin.

As mentioned previously and discussed in greater detail below, the invention also provides compositions and kits related to these methods. For example, compositions comprising a single first detection reagent and a plurality of subsets of particles (e.g., microspheres) are provided. The particles in each subset comprise a capture reagent specific for at least one of a plurality of protein kinases, and the particles in each subset are distinguishable from those of every other subset. The first detection reagent provides an indication of the presence of a phosphorylated amino acid residue (e.g., a phosphorylated serine, threonine and/or tyrosine). Optionally, the composition also includes the plurality of protein kinases; each kinase is optionally associated with one of the subsets of particles. As another example, kits, e.g., kits facilitating practice of the invention, are provided. For example, a kit comprising each of the components of the composition and instructions for using the composition to detect the presence or absence of the phosphorylated amino acid in a plurality of kinases, packaged in one or more containers, is a feature of the invention.

Detection of Nucleic Acid Binding Proteins

Another aspect of the present invention provides new methods, compositions, and kits for rapid and quantitative detection of one or more nucleic acid binding proteins in a single reaction. Thus, in one aspect, the invention includes methods of detecting the presence or absence of one or more nucleic acid binding proteins. In the methods, a sample comprising or suspected of comprising the one or more nucleic acid binding proteins is provided, along with one or more subsets of particles and one or more detection reagents. The particles in each subset comprise a nucleic acid binding site specific for at least one of the proteins (preferably, for one of the proteins), and the particles in each subset are distinguishable from those of every other subset. Each detection reagent provides an indication of the presence of at least one of the nucleic acid binding proteins. The one or more subsets of particles are exposed to the sample and then the one or more detection reagents are added to the exposed one or more subsets, or the one or more detection reagents are added to the sample and then the detection reagent(s) and the sample are exposed to the one or more subsets of particles. (This step permits the one or more proteins, if present, to associate with the binding site(s) and the detection reagent(s)). To determine whether each of the one or more proteins is present in the sample, each subset of particles is identified and the presence or absence of the one or more detection reagents is detected.

If one nucleic acid binding protein is to be detected, one subset of particles comprising a nucleic acid binding site for the protein and one detection reagent are typically provided. If two or more nucleic acid binding proteins are to be detected, two or more subsets of particles (each comprising a binding site specific for one of the proteins) are typically provided and pooled. In this example, either one or more than one detection reagent can be provided. For example, one detection reagent that recognizes a feature common to all the nucleic acid binding proteins can be provided. As another example, two or more detection reagents, each of which recognizes one of the nucleic acid binding proteins, can be provided. A combination of such strategies can also be used.

The particles can optionally be washed (e.g., with a solution comprising a buffer, salts, detergent and/or a blocking agent, or the like) at various steps, e.g., after addition of the sample to the particles and/or prior to detection of the detection reagent(s). The wash(es) can be included, e.g., for increased sensitivity and/or specificity, or omitted, e.g., for speed and simplicity.

In one class of embodiments, the particles are microspheres. In preferred embodiments, the particles are microspheres, and the microspheres of each subset are distinguishable from those of the other subsets on the basis of their fluorescent emission spectra and/or their diameter (i.e., their size).

The nucleic acid binding site specific for a particular protein can comprise essentially any sequence and type of nucleic acid that can be recognized and specifically bound by that protein. For example, the nucleic acid binding site can comprise single-stranded DNA, double-stranded DNA, single-stranded RNA and/or double-stranded RNA, as appropriate for the particular protein (e.g., a single-stranded, double-stranded or hairpin DNA or RNA oligonucleotide comprising a binding site for the protein). Appropriate binding sites for many proteins (particularly sequence-specific double-stranded DNA binding proteins) have been described in the literature, and an appropriate binding site can be determined for any sequence-specific nucleic acid binding protein by methods known in the art. For example, gel mobility shift assays and/or chemical or DNase footprinting can be used to identify a physiologically relevant binding site, or binding site selection can be performed to select a consensus high affinity binding site. See, e.g., Sambrook (infra), Ausubel (infra), Kosugi and Ohashi (2002) Plant J 30:337-348; Johannesson et al. (2001) Plant Mol Biol 45:63-73; Steadman et al. (2000) Nucleic Acids Res 28:2389-95; and Wolfe et al. (1999) J Mol Biol 285: 1917-34. The nucleic acid binding site can be covalently or noncovalently associated with the particles, as described in greater detail in the “Microspheres” section below. For example, an oligonucleotide comprising a free amino group (introduced during synthesis) can be covalently coupled to carboxylate-modified particles via a carbodiimide coupling method, or a biotinylated nucleic acid can be noncovalently associated with streptavidin-modified particles.

The one or more detection reagents can be essentially any molecule(s) capable of specifically recognizing one or more of the proteins. For example, the detection reagent(s) can comprise a nucleic acid (e.g., an oligonucleotide, an aptamer), a polypeptide (e.g., an antibody, a synthetic peptide, a recombinant protein), a substrate analog (e.g., a molecule that is a structural analog of an enzyme's substrate but that reacts very slowly or not at all and thus inhibits the enzyme by occupying its active site) and/or a small molecule (e.g., a ligand). In one embodiment, the one or more detection reagents comprise one or more antibodies specific for one or more of the nucleic acid binding proteins (e.g., one antibody for each protein, or one antibody for each family of related proteins, or a combination of protein-specific and family-specific antibodies).

Each detection reagent can be labeled and detected directly, or it can be indirectly detected. Thus, in one class of embodiments, the one or more detection reagents each comprises a first fluorescent label. In this class of embodiments, detecting the presence or absence of the detection reagent(s) comprises detecting a first fluorescent signal from the first label. In other embodiments, each detection reagent is not fluorescently labeled, but is instead detected by adding a labeled secondary agent that binds the detection reagent and detecting a signal from the labeled secondary agent. For example, the detection reagent can be biotinylated, and the secondary agent can be fluorescently labeled streptavidin. A combination of these strategies can be used, in which one detection reagent is detected directly and another indirectly. As noted for the above embodiments, fluorescent emission by the label(s) (whether on the detection reagent or on the secondary agent) is typically distinguishable from any fluorescent emission by the particles; many suitable fluorescent label-fluorescent particle combinations are possible. Fluorescent emission by the label can be conveniently detected, and subsets of particles can be identified, using, e.g., a flow cytometer or similar instrument. When multiple detection reagents are used to detect the presence of the proteins, the label for each of the detection reagents is typically but not necessarily the same.

The methods can be qualitative or quantitative. For example, the fluorescent signal from a detection reagent comprising a fluorescent label can be detected to indicate the presence or absence of the detection reagent and therefore of the corresponding nucleic acid binding protein(s), or the fluorescent signal can be quantitated to quantitate the protein(s). For example, microspheres that have captured nucleic acid binding proteins from a sample can be analyzed in parallel with control microsphere sets (e.g., microspheres exposed to known amounts of a control nucleic acid binding protein, e.g., a recombinant protein). One of skill can determine appropriate conditions for a quantitative assay by methods known in the art (e.g., using limiting concentrations of the proteins in the sample and non-limiting concentrations of nucleic acid binding sites and detection reagents, appropriate controls, and the like).

The nucleic acid binding proteins to be analyzed can be essentially any desired proteins. For example, the proteins can comprise an endogenous cellular protein or proteins (e.g., an intracellular protein, a plasma membrane protein and/or a secreted protein encoded by the cell's nuclear, mitochondrial and/or chloroplast genome) and/or a protein or proteins encoded by an infectious agent (e.g., a pathogenic virus, bacterium, protist, fungus or the like). Similarly, the sample comprising the proteins can be obtained or prepared from essentially any desired source. For example, the sample can be derived from an animal (e.g., a mammal, an invertebrate or an insect), a human, a plant, a cultured cell, and/or a microorganism. The sample can be derived, e.g., from a tissue, a biopsy or a tumor, e.g., from a human patient. The sample can comprise, for example, one or more of: a cell lysate (e.g., a lysate of cultured cells, a tissue lysate or a lysate of peripheral blood cells), an intercellular fluid, a conditioned culture medium or a bodily fluid (e.g., blood, serum, saliva, urine, sputum or spinal fluid).

The method can comprise additional steps. For example, at least one of the subsets of particles can be recovered, e.g., for additional analysis of the protein(s) associated with those particles. The particles can be recovered, for example, by being sorted into a separate sample tube by a flow cytometer and recovered by centrifugation (or by magnetic attraction if the particles are paramagnetic). The additional analysis can verify that the method is performing as expected. For example, a subset of particles can be recovered and analyzed, e.g., by mass spectroscopy, to determine if the intended protein and substantially only the intended protein was captured by the binding site. Alternatively, the additional analysis can provide new information. For example, the sample can be prepared and analyzed under mild conditions such that noncovalently associated protein complexes are not disrupted. In this example, a subset of particles can be recovered and analyzed, e.g., by mass spectroscopy or immunoassay, to determine what, if any, other proteins are associated with the nucleic acid binding protein captured by the binding site.

The method can be extended, providing further multiplexing capability with the addition of a second (and optional third, fourth, etc.) type of detection reagent. For example, an antibody (or other reagent) providing an indication of the presence of a posttranslational modification can be provided. As another example, an antibody to a protein that forms a complex with one of the nucleic acid binding proteins captured on the particles can be provided. For example, c-Jun can be captured with a DNA binding site comprising an AP-1 site and its presence can be detected with an anti-Jun antibody. Its phosphorylation state can be assessed with an anti-phosphoprotein antibody (phosphorylation of c-Jun increases its transcriptional activity). Other members of the AP-1 complex, of which Jun is a member, can be detected with other specific antibodies. Label configurations and the like as noted for the above embodiments apply here as well (e.g., the labels on these additional detection reagents are typically distinguishable from the label(s) on the detection reagents used to indicate presence of the proteins).

As previously noted, compositions and kits related to the methods are also features of the invention. For example, compositions comprising one or more subsets of particles (e.g., microspheres) are provided. The particles in each subset comprise a nucleic acid binding site specific for at least one nucleic acid binding protein, and the particles in each subset are distinguishable from those of every other subset. The composition optionally also includes one or more nucleic acid binding proteins; each protein is optionally associated with one of the one or more subsets of particles. As another example, kits, e.g., kits facilitating practice of the invention, are provided. For example, a kit comprising each of the components of the composition and instructions for using the composition to detect at least one nucleic acid binding protein, packaged in one or more containers, is a feature of the invention. The kits and compositions are discussed in greater detail below.

Detection of Posttranslational Modifications: Array Assay

In another aspect, the invention includes methods of and compositions and kits for detecting the presence or absence of a plurality of posttranslational modifications of a plurality of proteins in a sample. In the methods, the sample comprising the proteins is provided, along with a plurality of detection reagents and a solid support comprising a plurality of capture reagents. Each capture reagent is specific for at least one of the proteins (preferably, for one of the proteins), and each capture reagent is provided at a known, pre-determined position on the solid support. That is, the capture reagents form an array, such that each capture reagent (and thus, the protein bound to each capture reagent) can be identified by the position at which it is immobilized. Each detection reagent provides an indication of the presence of one of the posttranslational modifications. The proteins are bound to the capture reagents and exposed to the detection reagents. The presence or absence of each of the detection reagents is detected (at each position) to determine whether each of the proteins comprises each of the posttranslational modifications.

The posttranslational modifications can be essentially any modifications. Examples of posttranslational modifications that can be detected include, but are not limited to, phosphorylation (e.g., phosphorylation of a serine, threonine and/or tyrosine residue), ubiquitination, sumoylation, glycosylation, prenylation, myristoylation, farnesylation, attachment of a fatty acid, attachment of a GPI anchor, acetylation, methylation and nucleotidylation (e.g., ADP-ribosylation).

Binding of the proteins to the capture reagents and exposure of the proteins to the detection reagents can occur simultaneously or sequentially, in various orders. For example, in one embodiment, the support is exposed to the sample, and the detection reagents are added to the exposed support. In this embodiment, the exposed support is optionally washed prior to the addition of the detection reagents (e.g., with a solution comprising a buffer, salts, detergent and/or a blocking agent, or the like). As another example, in another embodiment the sample and the detection reagents are combined and then added to the support. The support can optionally be washed prior to detection of the detection reagents. The wash(es) can be included, e.g., for increased sensitivity and/or specificity, or omitted, e.g., for speed and simplicity.

In one class of embodiments, the solid support is a membrane (e.g., a nylon, PVDF, or nitrocellulose membrane), a plate (e.g., glass or plastic), or a slide (e.g., glass or plastic). Other supports, e.g., other basically two-dimensional supports, can also be used.

The capture reagent for a particular protein can be essentially any molecule that binds specifically to that protein. For example, a capture reagent can comprise a nucleic acid (e.g., an oligonucleotide, a nucleic acid binding site, an aptamer), a polypeptide (e.g., an antibody, a recombinant protein, a synthetic peptide), a substrate analog (e.g., a molecule that is a structural analog of an enzyme's substrate but that reacts very slowly or not at all and thus inhibits the enzyme by occupying its active site) and/or a small molecule (e.g., a ligand). A single position on the support typically (but not necessarily) comprises a single type of capture reagent, while different positions can comprise the same or different types of capture reagents. For example, one position can comprise an antibody specific for a first protein while a second position comprises an antibody specific for a second protein, or one position can comprise an antibody specific for a first protein while a second position comprises a single-stranded or double-stranded oligonucleotide binding site for a second protein. The capture reagents can be covalently or noncovalently associated with the solid support. For example, the capture reagents can be adsorbed to a membrane or covalently coupled to an aldehyde-coated slide. As another example, biotinylated capture reagents can be noncovalently associated with streptavidin-printed positions on a support, or antibody capture reagents can be noncovalently associated with protein A or G-printed positions on a support.

Similarly, the detection reagents can be essentially any molecules capable of specifically recognizing the posttranslational modifications. For example, the detection reagents can comprise a nucleic acid (e.g., an oligonucleotide, an aptamer), a polypeptide (e.g., an antibody, a synthetic peptide, a recombinant protein, e.g., a recombinant protein comprising an SH2, PTB, 14-3-3, FHA, WD40 and/or WW domain capable of binding a phosphorylated residue or peptide) and/or a small molecule. In one class of embodiments, the detection reagents comprise one or more of: an antibody specific for a phosphorylated tyrosine, serine and/or threonine residue, an antibody specific for ubiquitin, an antibody specific for a SUMO polypeptide, an antibody specific for a carbohydrate moiety, an antibody specific for an acetyl group, an antibody specific for a prenyl group, or the like. As another example, the detection reagents can comprise one or more lectins.

Each detection reagent can be labeled and detected directly, or it can be indirectly detected. Thus, in one class of embodiments, each detection reagent comprises a fluorescent label emitting a distinct signal (e.g., one detection reagent can be labeled with fluorescein and another with PC5, or one detection reagent can be labeled with Cy3 and another with Cy5; many suitable combinations are known in the art, and selection of an appropriate combination for a particular application is routine for one of skill). In this class of embodiments, detecting the presence or absence of the detection reagents comprises detecting fluorescent signals from the labels. In other embodiments, the detection reagents are not fluorescently labeled, but are instead detected by adding a labeled secondary agent that binds the detection reagents and detecting a signal from the labeled secondary agent. For example, one of the detection reagents can be biotinylated, and the secondary agent can be fluorescently labeled streptavidin. A combination of these strategies can be used, in which one detection reagent is detected directly and another indirectly. Fluorescent emissions by the labels can be conveniently detected, e.g., using a commercially available instrument such as an ArrayWORx fluorescence slide scanner (Applied Precision, Issaquah, Wash.) or a GenePix 4000A microarray scanner (Axon Instruments, Foster City, Calif.). Details regarding labels and detection strategies can be found, e.g., in The Handbook of Fluorescent Probes and Research Products, Ninth Edition by Richard P. Haughland, published by Molecular Probes, Inc. The Handbook is available in print from Molecular Probes, or on-line on the world wide web at www.molecularprobes.com.

The methods can be qualitative or quantitative. For example, the fluorescent signal from each detection reagent that comprises a fluorescent label can be detected to indicate the presence or absence of the detection reagent and therefore of the corresponding posttranslational modification, or each fluorescent signal can be quantitated to provide an indication of the extent of modification. One of skill can determine appropriate conditions for a quantitative assay by methods known in the art (e.g., using limiting concentrations of the proteins in the sample and non-limiting concentrations of capture and detection reagents, appropriate controls, and the like).

As for the above embodiments, the proteins to be analyzed can be essentially any desired proteins. For example, the proteins can comprise an endogenous cellular protein or proteins (e.g., an intracellular protein, a plasma membrane protein and/or a secreted protein encoded by the cell's nuclear, mitochondrial and/or chloroplast genome) and/or a protein or proteins encoded by an infectious agent (e.g., a pathogenic virus, bacterium, protist, fungus or the like). In one embodiment, the plurality of proteins comprises a plurality of protein kinases. Similarly, the sample comprising the proteins can be obtained or prepared from essentially any desired source. For example, the sample can be derived from an animal (e.g., a mammal, an invertebrate or an insect), a human, a plant, a cultured cell, and/or a microorganism. The sample can be derived, e.g., from a tissue, a biopsy or a tumor, e.g., from a human patient. The sample can comprise, for example, one or more of: a cell lysate (e.g., a lysate of cultured cells, a tissue lysate or a lysate of peripheral blood cells), an intercellular fluid, a conditioned culture medium or a bodily fluid (e.g., blood, serum, saliva, urine, sputum or spinal fluid).

As mentioned previously and discussed in greater detail below, the invention also provides compositions and kits related to these methods. For example, compositions comprising a plurality of proteins comprising or suspected of comprising a plurality of posttranslational modifications, a solid support comprising a plurality of capture reagents, and a plurality of detection reagents are provided. Each capture reagent is specific for at least one of the proteins, and each capture reagent is provided at a known, pre-determined position on the solid support. Each capture reagent (and thus, the protein bound to each capture reagent) can thus be identified by the position at which it is immobilized. Each detection reagent provides an indication of the presence of one of the posttranslational modifications. As another example, kits, e.g., kits facilitating practice of the invention, are provided. For example, a kit comprising each of the components of the composition and instructions for using the composition to detect a plurality of posttranslational modifications, packaged in one or more containers, is a feature of the invention.

Additional Details Regarding Compositions, Systems and Kits

Compositions Related to Detection of Posttranslational Modification: Particle Assay

As noted above, compositions, systems and kits that can be used in practicing the methods of the invention are also features of the invention. Thus, one general class of embodiments provides a composition comprising a single first detection reagent and a plurality of subsets of particles. The particles in each subset comprise a capture reagent specific for at least one of a plurality of proteins comprising or suspected of comprising a first posttranslational modification, and the particles in each subset are distinguishable from those of every other subset. The first detection reagent provides an indication of the presence of the first posttranslational modification. Preferably, the particles in each subset comprise a capture reagent specific for one of the plurality of proteins.

The composition optionally also includes the plurality of proteins comprising or suspected of comprising the first posttranslational modification. Optionally, each of the plurality of proteins is associated with one of the subsets of particles (typically, via noncovalent association with the capture reagent). The proteins to be analyzed can be essentially any desired proteins. For example, the proteins can comprise an endogenous cellular protein or proteins (e.g., an intracellular protein, a plasma membrane protein and/or a secreted protein encoded by the cell's nuclear, mitochondrial and/or chloroplast genome) and/or a protein or proteins encoded by an infectious agent (e.g., a pathogenic virus, bacterium, protist, fungus or the like). In one embodiment, the plurality of proteins comprises a plurality of protein kinases. Similarly, the proteins can be obtained or prepared from essentially any desired source. For example, the proteins can be derived from an animal (e.g., a mammal, an invertebrate or an insect), a human, a plant, a cultured cell, and/or a microorganism. The proteins can be derived, e.g., from a tissue, a biopsy or a tumor, e.g., from a human patient. The proteins can be obtained, for example, from a cell lysate (e.g., a lysate of cultured cells, a tissue lysate or a lysate of peripheral blood cells), an intercellular fluid, a conditioned culture medium and/or a bodily fluid (e.g., blood, serum, saliva, urine, sputum or spinal fluid).

The first posttranslational modification can be essentially any modification. For example, the first posttranslational modification can be phosphorylation, e.g., phosphorylation of a serine, threonine and/or tyrosine residue. Other examples of posttranslational modifications include, but are not limited to, ubiquitination, sumoylation, glycosylation, prenylation, myristoylation, farnesylation, attachment of a fatty acid, attachment of a GPI anchor, acetylation, methylation and nucleotidylation (e.g., ADP-ribosylation).

In one class of embodiments, the particles are microspheres. In preferred embodiments, the particles are microspheres, and the microspheres of each subset are distinguishable from those of the other subsets on the basis of their fluorescent emission spectra and/or their diameter (i.e., their size).

The capture reagent for a particular protein can be essentially any molecule that binds specifically to that protein. For example, a capture reagent can comprise a nucleic acid (e.g., an oligonucleotide, a nucleic acid binding site, an aptamer), a polypeptide (e.g., an antibody, a recombinant protein, a synthetic peptide), a substrate analog (e.g., a molecule that is a structural analog of an enzyme's substrate but that reacts very slowly or not at all and thus inhibits the enzyme by occupying its active site) and/or a small molecule (e.g., a ligand). A single subset of particles typically (but not necessarily) comprises a single type of capture reagent, while different subsets can comprise the same or different types of capture reagents. For example, one subset can comprise an antibody specific for a first protein while a second subset comprises an antibody specific for a second protein, or one subset can comprise an antibody specific for a first protein while a second subset comprises a single-stranded or double-stranded oligonucleotide binding site for a second protein. The capture reagent can be covalently or noncovalently associated with the particles, as described in greater detail in the “Microspheres” section below. For example, the capture reagent can be covalently coupled to carboxylate-modified particles via a carbodiimide coupling method or to maleimide-modified particles via a thiol-maleimide interaction. As another example, a biotinylated capture reagent can be noncovalently associated with streptavidin-modified particles, or a GST-tagged or polyhistidine-tagged recombinant protein can be noncovalently associated with glutathione or Ni²⁺ coated particles.

Similarly, the first detection reagent can be essentially any molecule capable of specifically recognizing the first posttranslational modification. For example, the first detection reagent can comprise a nucleic acid (e.g., an oligonucleotide, an aptamer), a polypeptide (e.g., an antibody, a synthetic peptide, a recombinant protein, e.g., a recombinant protein comprising an SH2, PTB, 14-3-3, FHA, WD40 and/or WW domain capable of binding a phosphorylated residue or peptide) and/or a small molecule. In one class of embodiments, the first detection reagent is an antibody specific for a phosphorylated tyrosine, serine and/or threonine residue (e.g., a monoclonal antibody against phosphoserine, phosphothreonine or phosphotyrosine, a polyclonal antibody against phosphothreonine and phosphoserine, or a polyclonal antibody against phosphotyrosine, among many other possible examples). In other embodiments, the first detection reagent is an antibody specific for another posttranslational modification; for example, an antibody specific for ubiquitin, SUMO, a carbohydrate moiety, an acetyl group, a prenyl group, or the like. In other embodiments, the first detection reagent is a lectin.

The first detection reagent can comprise a first fluorescent label. Alternatively, the composition can further comprise a labeled secondary agent that binds the first detection reagent. For example, the first detection reagent can be biotinylated, and the secondary agent can be fluorescently labeled streptavidin. As noted for the embodiments above, fluorescent emission by the first label (whether on the first detection reagent or on the secondary agent) is typically distinguishable from any fluorescent emission by the particles.

The composition can optionally include a second detection reagent. The second detection reagent can provide an indication of the presence of a second posttranslational modification. To list only a few of the possible examples, the first detection reagent can be specific for tyrosine phosphorylation and the second for serine phosphorylation, the first detection reagent can be specific for phosphorylation (tyrosine, threonine and/or serine) and the second for ubiquitination, or the first detection reagent can be specific for glycosylation and the second for ubiquitination. Alternatively, the second detection reagent can provide an indication of the presence of a specific protein (e.g., the second detection reagent can be an antibody to a protein that forms a complex with one of the proteins captured by the capture reagents), protein family, or the like. Like the first detection reagent, the second detection reagent can itself be labeled, or it can be indirectly detected by use of a secondary agent. The label for the second detection reagent is typically distinguishable from that for the first detection reagent (and from the particles, if applicable).

In one aspect, systems comprising the compositions noted above and, e.g., components such as fluid or particle handling elements, fluid or particle containing elements, lasers, detectors, and/or the like are a feature of the invention.

Kits (e.g., a kit comprising each of the components of the composition and instructions for using the composition to detect at least one posttranslational modification, packaged in one or more containers) form another feature of the invention. One general class of embodiments provides a kit for detecting the presence or absence of a first posttranslational modification of a plurality of proteins in a sample. The kit comprises a plurality of subsets of particles, the particles in each subset being distinguishable from those of every other subset, and a single first detection reagent capable of providing an indication of the presence of the first posttranslational modification, packaged in one or more containers. In one class of embodiments, the particles in each subset comprise a capture reagent specific for at least one of the proteins (preferably, for one of the proteins). Alternatively, in other embodiments, the particles in each subset are capable of being associated with a capture reagent supplied by a user of the kit.

All of the various features for the compositions noted above apply here as well, e.g., for types of capture and detection reagents, microspheres, label configurations, optional second detection reagent, and the like. The proteins to be analyzed can be essentially any proteins, from essentially any source. In one embodiment, the proteins are protein kinases, and each capture reagent is specific for one of the kinases.

The kit typically also includes instructions for use of the kit; for example, instructions for covalently or noncovalently attaching a capture reagent to each subset of particles if the capture reagent is not already attached, instructions for binding the proteins to the capture reagents, instructions for exposing the proteins to the first detection reagent and/or instructions for determining whether each of the proteins comprises the first posttranslational modification by identifying each subset of particles and detecting the presence or absence of the first detection reagent. The kit can also include a buffer solution, a blocking agent, controls (known proteins with and without the posttranslational modification) and/or the like.

In one class of embodiments, the kit can be used for diagnosis, prognosis or monitoring of disease by detecting the phosphorylation state of protein kinases. In this class of embodiments, the proteins to be analyzed are protein kinases, the capture reagent on each subset of particles is specific for one of the kinases, and the first detection reagent is specific for phosphorylated serine, threonine and/or tyrosine. The kit can also comprise control kinase activity profiles or samples for generating such profiles.

Compositions Related to Detection of Nucleic Acid Binding Proteins

Another general class of embodiments provides a composition comprising one or more subsets of particles; the particles in each subset comprise a nucleic acid binding site specific for at least one nucleic acid binding protein (preferably, for one nucleic acid binding protein), and the particles in each subset are distinguishable from those of every other subset. The composition optionally also includes one or more nucleic acid binding proteins. Optionally, each nucleic acid binding protein is associated with one of the one or more subsets of particles.

The proteins can be essentially any desired proteins, obtained or prepared from essentially any desired source. For example, the proteins can comprise an endogenous cellular protein or proteins (e.g., an intracellular protein, a plasma membrane protein and/or a secreted protein encoded by the cell's nuclear, mitochondrial and/or chloroplast genome) and/or a protein or proteins encoded by an infectious agent (e.g., a pathogenic virus, bacterium, protist, fungus or the like). They can be derived, e.g., from an animal (e.g., a mammal, an invertebrate or an insect), a human, a plant, a cultured cell, and/or a microorganism. The proteins can be obtained, e.g., from a tissue, a biopsy, a tumor (e.g., from a human patient), a cell lysate (e.g., a lysate of cultured cells, a tissue lysate or a lysate of peripheral blood cells), an intercellular fluid, a conditioned culture medium and/or a bodily fluid (e.g., blood, serum, saliva, urine, sputum or spinal fluid).

In one class of embodiments, the particles are microspheres. In preferred embodiments, the particles are microspheres, and the microspheres of each subset are distinguishable from those of the other subsets on the basis of their fluorescent emission spectra and/or their diameter (i.e., their size).

The nucleic acid binding site specific for a particular protein can comprise essentially any sequence and type of nucleic acid that can be recognized and specifically bound by that protein. For example, the nucleic acid binding site can comprise single-stranded DNA, double-stranded DNA, single-stranded RNA and/or double-stranded RNA, as appropriate for the particular protein (e.g., a single-stranded, double-stranded or hairpin DNA or RNA oligonucleotide comprising a binding site for the protein). An appropriate binding site for a given protein can be determined as noted above. The nucleic acid binding site can be covalently or noncovalently associated with the particles, as described in greater detail in the “Microspheres” section below. For example, an oligonucleotide comprising a free amino group (introduced during synthesis) can be covalently coupled to carboxylate-modified particles via a carbodiimide coupling method, or a biotinylated nucleic acid can be noncovalently associated with streptavidin-modified particles.

The composition can optionally include one or more detection reagents, each of which provides an indication of the presence of at least one nucleic acid binding protein. The one or more detection reagents can be essentially any molecule(s) capable of specifically recognizing one or more of the proteins. For example, the detection reagent(s) can comprise a nucleic acid (e.g., an oligonucleotide, an aptamer), a polypeptide (e.g., an antibody, a synthetic peptide, a recombinant protein), a substrate analog (e.g., a molecule that is a structural analog of an enzyme's substrate but that reacts very slowly or not at all and thus inhibits the enzyme by occupying its active site) and/or a small molecule (e.g., a ligand). In one embodiment, the one or more detection reagents comprise one or more antibodies specific for one or more of the nucleic acid binding proteins (e.g., one antibody for each protein, or one antibody for each family of related proteins, or a combination of protein-specific and family-specific antibodies).

Each detection reagent can be labeled and detected directly, or it can be indirectly detected. Thus, in one class of embodiments, the one or more detection reagents each comprises a first fluorescent label. In other embodiments, the composition further comprises a labeled secondary agent that binds the one or more detection reagents. For example, the detection reagent can be biotinylated, and the secondary agent can be fluorescently labeled streptavidin. In other embodiments, a combination of these strategies is used; one detection reagent can comprise a fluorescent label, while a labeled secondary agent binds another detection reagent. As noted for the above embodiments, fluorescent emission by the label(s) (whether on the detection reagent or on the secondary agent) is typically distinguishable from any fluorescent emission by the particles; many suitable fluorescent label-fluorescent particle combinations are possible. When multiple detection reagents are used to detect the presence of the proteins, the label for each of the detection reagents is typically but not necessarily the same.

Optionally, the composition further comprises a second (and optional third, fourth, etc.) type of detection reagent. For example, an antibody (or other reagent) providing an indication of the presence of a posttranslational modification can be provided. As another example, an antibody to a protein that forms a complex with one of the nucleic acid binding proteins captured on the particles can be provided. Label configurations and the like as noted for the above embodiments apply here as well (e.g., the labels on these additional detection reagents are typically distinguishable from the label(s) on the detection reagents used to indicate presence of the proteins and from the particles).

In one aspect, systems comprising the compositions noted above and, e.g., components such as fluid or particle handling elements, fluid or particle containing elements, lasers, detectors, and/or the like are a feature of the invention.

Kits, (e.g., a kit comprising each of the components of the composition and instructions for using the composition to detect at least one nucleic acid binding protein, packaged in one or more containers) form another feature of the invention. One general class of embodiments provides a kit for detecting the presence or absence of one or more nucleic acid binding proteins in a sample. The kit comprises one or more subsets of particles and one or more detection reagents, packaged in one or more containers. The particles in each subset are distinguishable from those of every other subset. Each detection reagent provides an indication of the presence of at least one of the nucleic acid binding proteins. In one class of embodiments, the particles in each subset comprise a nucleic acid binding site specific for at least one of the nucleic acid binding proteins (preferably, for one of the nucleic acid binding proteins). Alternatively, in other embodiments, the particles in each subset are capable of being associated with a nucleic acid binding site supplied by a user of the kit.

All of the various features for the compositions noted above apply here as well, e.g., for types of binding sites and detection reagents, microspheres, label configurations, optional second detection reagent, and the like.

The kit typically also includes instructions for use of the kit; for example, instructions for attaching a nucleic acid binding site to each subset of particles, if the binding site is not already attached, instructions for exposing the one or more subsets of particles to a sample and adding the one or more detection reagents to the exposed subsets and/or instructions for determining whether each of the proteins is present in the sample by identifying each subset of particles and detecting the presence or absence of the one or more detection reagents. The kit can also include a buffer solution, a blocking agent, controls and/or the like.

Compositions Related to Detection of Posttranslational Modifications: Array Assay

Another general class of embodiments provides a composition comprising a plurality of proteins comprising or suspected of comprising a plurality of posttranslational modifications, a solid support comprising a plurality of capture reagents, and a plurality of detection reagents. Each capture reagent is specific for at least one of the proteins (preferably, for one of the proteins), and each capture reagent is provided at a known, pre-determined position on the solid support. That is, the capture reagents form an array, such that each capture reagent (and thus, the protein bound to each capture reagent) can be identified by the position at which it is immobilized. Each detection reagent provides an indication of the presence of one of the posttranslational modifications.

The posttranslational modifications can be essentially any modifications. Examples of posttranslational modifications include, but are not limited to, phosphorylation (e.g., phosphorylation of a serine, threonine and/or tyrosine residue), ubiquitination, sumoylation, glycosylation, prenylation, myristoylation, farnesylation, attachment of a fatty acid, attachment of a GPI anchor, acetylation, methylation and nucleotidylation (e.g., ADP-ribosylation).

In one class of embodiments, the solid support is a membrane (e.g., a nylon, PVDF, or nitrocellulose membrane), a plate (e.g., glass or plastic), or a slide (e.g., glass or plastic). Other supports, e.g., other basically two-dimensional supports, can also be used.

The capture reagent for a particular protein can be essentially any molecule that binds specifically to that protein. For example, a capture reagent can comprise a nucleic acid (e.g., an oligonucleotide, a nucleic acid binding site, an aptamer), a polypeptide (e.g., an antibody, a recombinant protein, a synthetic peptide), a substrate analog (e.g., a molecule that is a structural analog of an enzyme's substrate but that reacts very slowly or not at all and thus inhibits the enzyme by occupying its active site) and/or a small molecule (e.g., a ligand). A single position on the support typically (but not necessarily) comprises a single type of capture reagent, while different positions can comprise the same or different types of capture reagents. For example, one position can comprise an antibody specific for a first protein while a second position comprises an antibody specific for a second protein, or one position can comprise an antibody specific for a first protein while a second position comprises a single-stranded or double-stranded oligonucleotide binding site for a second protein. The capture reagents can be covalently or noncovalently associated with the solid support. For example, the capture reagents can be adsorbed to a membrane or covalently coupled to an aldehyde-coated slide. As another example, biotinylated capture reagents can be noncovalently associated with streptavidin-printed positions on a support, or antibody capture reagents can be noncovalently associated with protein A or G-printed positions on a support.

Similarly, the detection reagents can be essentially any molecules capable of specifically recognizing the posttranslational modifications. For example, the detection reagents can comprise a nucleic acid (e.g., an oligonucleotide, an aptamer), a polypeptide (e.g., an antibody, a synthetic peptide, a recombinant protein, e.g., a recombinant protein comprising an SH2, PTB, 14-3-3, FHA, WD40 and/or WW domain capable of binding a phosphorylated residue or peptide) and/or a small molecule. In one class of embodiments, the detection reagents comprise one or more of: an antibody specific for a phosphorylated tyrosine, serine and/or threonine residue, an antibody specific for ubiquitin, an antibody specific for SUMO, an antibody specific for a carbohydrate moiety, an antibody specific for an acetyl group, an antibody specific for a prenyl group, or the like. As another example, the detection reagents can comprise one or more lectins.

Each detection reagent can be labeled and detected directly, or it can be indirectly detected. Thus, in one class of embodiments, each detection reagent comprises a fluorescent label emitting a distinct signal (e.g., one detection reagent can be labeled with fluorescein and another with PC5, or one detection reagent can be labeled with Cy3 and another with Cy5; many suitable combinations are known in the art, and selection of an appropriate combination for a particular application is routine for one of skill). In other embodiments, the detection reagents are not fluorescently labeled, but instead the composition comprises one or more labeled secondary agents that bind the detection reagents. For example, one of the detection reagents can be biotinylated, and one of the secondary agents can be fluorescently labeled streptavidin. A combination of these strategies can be used, in which one detection reagent is detected directly and another indirectly.

In one aspect, systems comprising the compositions noted above and, e.g., components such as lasers, detectors, and/or the like are a feature of the invention.

Kits (e.g., a kit comprising each of the components of the composition and instructions for using the composition to detect a plurality of posttranslational modifications, packaged in one or more containers) form another feature of the invention. One general class of embodiments provides a kit for detecting the presence or absence of a plurality of posttranslational modifications of a plurality of proteins in a sample. The kit comprises a solid support comprising a plurality of capture reagents and a plurality of detection reagents, packaged in one or more containers. Each capture reagent is specific for at least one of the proteins (preferably, for one of the proteins), and each capture reagent is provided at a known, pre-determined position on the solid support. Each detection reagent provides an indication of the presence of one of the posttranslational modifications.

All of the various features for the compositions noted above apply here as well, e.g., for types of capture and detection reagents, posttranslational modifications, label configurations, support types, and the like. The proteins to be analyzed can be essentially any proteins, from essentially any source.

The kit typically also includes instructions for use of the kit; for example, instructions for binding the proteins to the capture reagents, instructions for exposing the proteins to the detection reagents, and/or instructions for determining whether each of the proteins comprises the posttranslational modifications by identifying each position on the support and detecting the presence or absence of each detection reagent. The kit can also include a buffer solution, a blocking agent, controls (known proteins with and without the modifications), and/or the like.

Systems

In one aspect, the invention includes systems, e.g., systems used to practice the methods herein and/or comprising the compositions described herein. The system can include, e.g., a fluid and/or particle handling element, a fluid and/or particle containing element, a laser for exciting a fluorescent label, a detector for detecting fluorescent emissions from the fluorescent label and/or a robotic element that moves other components of the system from place to place as needed. For example, in one class of embodiments, a composition of the invention is contained in a flow cytometer, a Luminex 100™ or HTS™ instrument, a microplate reader, a slide scanner, a microarray scanner, or like instrument.

The system can optionally include a computer. The computer can include appropriate software for receiving user instructions, either in the form of user input into a set of parameter fields, e.g., in a GUI, or in the form of preprogrammed instructions, e.g., preprogrammed for a variety of different specific operations. The software optionally converts these instructions to appropriate language for controlling the operation of components of the system (e.g., for controlling a fluid handling element, robotic element and/or laser). The computer can also receive data from other components of the system, e.g., from a detector, and can interpret the data, provide it to a user in a human readable format, or use that data to initiate further operations, in accordance with any programming by the user.

Fluorescent Labels

The compositions of this invention optionally include one or more labels, typically, fluorescent labels. A number of fluorescent labels are well known in the art, including but not limited to, hydrophobic fluorophores (e.g., rhodamine, phycoerythrin, Alexa Fluor 488 and fluorescein), green fluorescent protein (GFP) and variants thereof (e.g., cyan fluorescent protein and yellow fluorescent protein) and quantum dots. See, e.g., Handbook of Fluorescent Probes and Research Products, Ninth Edition or Web Edition, from Molecular Probes, Inc. for descriptions of fluorophores emitting at various different wavelengths (including tandem conjugates of fluorophores that can facilitate simultaneous excitation and detection of multiple labeled species). For use of quantum dots as labels for biomolecules, see, e.g., Dubertret et al. (2002) Science 298:1759; Nature Biotechnology (2003) 21:41-46; and Nature Biotechnology (2003) 21:47-51.

Labels can be introduced to molecules, e.g., polypeptides, nucleic acids and small molecules, during synthesis or by postsynthetic reactions by techniques established in the art; for example, kits for fluorescently labeling proteins, antibodies, nucleic acids, DNA and oligonucleotides with various fluorophores are available from Molecular Probes, Inc. (www.molecularprobes.com). Similarly, signals from the labels (e.g., absorption by and/or fluorescent emission from a fluorescent label) can be detected by essentially any method known in the art. For example, multicolor detection, detection of FRET, fluorescence polarization, and the like, are well known in the art.

Molecular Biological Techniques

In practicing the present invention, many conventional techniques in molecular biology, microbiology, and recombinant DNA technology are optionally used. These techniques are well known and are explained in, for example, Berger and Kimmel, Guide to Molecular Cloning Techniques, Methods in Enzymology volume 152 Academic Press, Inc., San Diego, Calif. (Berger); Sambrook et al., Molecular Cloning—A Laboratory Manual (3rd Ed.), Vol. 1-3, Cold Spring Harbor Laboratory, Cold Spring Harbor, N.Y., 2000 (“Sambrook”) and Current Protocols in Molecular Biology, F. M. Ausubel et al., eds., Current Protocols, a joint venture between Greene Publishing Associates, Inc. and John Wiley & Sons, Inc. (supplemented through 2002) (“Ausubel”). Other useful references, e.g., for cell isolation and culture (e.g., for subsequent nucleic acid or protein isolation) include Freshney (1994) Culture of Animal Cells, a Manual of Basic Technique, third edition, Wiley-Liss, New York and the references cited therein; Payne et al. (1992) Plant Cell and Tissue Culture in Liquid Systems John Wiley & Sons, Inc. New York, N.Y.; Gamborg and Phillips (Eds.) (1995) Plant Cell, Tissue and Organ Culture; Fundamental Methods Springer Lab Manual, Springer-Verlag (Berlin Heidelberg New York) and Atlas and Parks (Eds.) The Handbook of Microbiological Media (1993) CRC Press, Boca Raton, Fla.

Making Nucleic Acids

Methods of making nucleic acids (e.g., by in vitro amplification, purification from cells or chemical synthesis), methods for manipulating nucleic acids (e.g., by restriction enzyme digestion, ligation, etc.) and various vectors, cell lines and the like useful in manipulating and making nucleic acids are described in the above references.

In addition, essentially any nucleic acid (including, e.g., labeled or biotinylated oligonucleotides) can be custom or standard ordered from any of a variety of commercial sources, such as The Midland Certified Reagent Company (www.mcrc.com), The Great American Gene Company (www.genco.com), ExpressGen Inc. (www.expressgen.com), QIAGEN (http://oligos.qiagen.com) and many others.

A label, biotin, or other moiety can optionally be introduced to a nucleic acid, either during or after synthesis. For example, a biotin phosphoramidite can be incorporated during chemical synthesis of an oligonucleotide. Alternatively, any nucleic acid can be biotinylated using techniques known in the art; suitable reagents are commercially available, e.g., from Pierce Biotechnology (www.piercenet.com). Similarly, any nucleic acid can be fluorescently labeled, for example, by using commercially available kits such as those from Molecular Probes, Inc. (www.molecularprobes.com) or Pierce Biotechnology (www.piercenet.com).

Aptamers

An aptamer is a nucleic acid capable of interacting with a binding partner, such as a protein, peptide or nucleic acid. Interaction with a nucleic acid ligand includes interactions other than complementary base pairing along the length of the aptamer and the nucleic acid ligand. An aptamer can be, e.g., a DNA or RNA, and can be, e.g., a chemically synthesized oligonucleotide. Aptamers can be selected, designed, etc. for binding various molecules by methods known in the art. For example, aptamers are reviewed in Sun S. (2000) “Technology evaluation: SELEX, Gilead Sciences Inc.” Curr Opin Mol Ther. 2: 100-5; Patel D J, Suri A K. (2000) “Structure, recognition and discrimination in RNA aptamer complexes with cofactors, amino acids, drugs and aminoglycoside antibiotics” J Biotechnol. 74:39-60; Brody E N, Gold L. (2000) “Aptamers as therapeutic and diagnostic agents” J Biotechnol. 74:5-13; Hermann T, Patel D J. (2000) “Adaptive recognition by nucleic acid aptamers” Science 287:820-5; Jayasena S D. (1999) “Aptamers: an emerging class of molecules that rival antibodies in diagnostics” Clin Chem. 45:1628-50; and Famulok M, Mayer G. (1999) “Aptamers as tools in molecular biology and immunology” Curr Top Microbiol Immunol. 243:123-36.

Making Polypeptides

Polypeptides (e.g., for use as capture or detection reagents, or for use in raising antibodies) can be obtained by any of a variety of methods known in the art. For example, smaller peptides (e.g., less than 50 amino acids long) are conveniently synthesized by standard chemical techniques and can optionally be chemically or enzymatically ligated to form larger polypeptides. Peptides (including, e.g., fluorescently labeled or biotinylated peptides) can also be custom ordered from a variety of commercial sources, including Biopeptide Co., LLC (www.peptide-synthesis.com), QIAGEN, Inc. (www.merlincustomservices.com) and Research Genetics (www.resgen.com). As another example, RNA encoding the polypeptide can be chemically synthesized (see, e.g., Oligonucleotide Synthesis (1984) Gait ed., IRL Press, Oxford). As yet another example, polypeptides can be purified from biological sources by methods well known in the art; polypeptides can be purified from a natural source or can optionally be produced in their naturally occurring, truncated, or fusion protein forms by recombinant DNA technology using techniques well known in the art (e.g., in vitro recombinant DNA techniques, synthetic techniques and in vivo genetic recombination), e.g., as described in the references above.

In brief, a polypeptide (e.g., a protein, a protein domain, a fusion protein) can be expressed in and purified from a suitable host cell. Expression occurs by placing a nucleotide sequence encoding the polypeptide into an appropriate expression vector, introducing the resulting expression vector into a suitable host cell and culturing the transformed host cell under conditions suitable for expression of the polypeptide; the recombinant polypeptide can then be purified from the host cell. Appropriate expression vectors are known in the art. For example, pET-14b, pcDNA1Amp, and pVL1392 are available from Novagen (www.novagen.com) and Invitrogen (www.invitrogen.com) and are suitable vectors for expression in E. coli, COS cells and baculovirus-infected insect cells, respectively. These vectors are illustrative of those that are known in the art. Suitable host cells can be any cell capable of growth in a suitable media and allowing purification of the expressed protein. Examples of suitable host cells include bacterial cells, such as E. coli, Streptococci, Staphylococci, Streptomyces and Bacillus subtilis cells; fungal cells such as yeast cells, e.g., Saccharomyces or Pichia, and Aspergillus cells; insect cells such as Drosophila S2 and Spodoptera Sf9 cells, mammalian cells such as CHO, COS, HeLa; and plant cells. Culturing and growth of the transformed host cells can occur under conditions that are known in the art (see, e.g., the references previously noted). The conditions (e.g., temperature and chemicals) will generally depend upon the host cell and the type of vector and promoter used.

Purification of the polypeptide can be accomplished using standard procedures known to and used by those of skill in the art. Generally, the transformed cells expressing the polypeptide are broken and crude purification is performed to remove debris and some contaminating proteins, followed by further purification (e.g., by chromatography) to the desired level of purity. Cells can be broken by known techniques such as homogenization, sonication, detergent lysis and freeze-thaw techniques. The polypeptide can be recovered and purified (partially or substantially to homogeneity) by any of a number of methods well known in the art, including, e.g., ammonium sulfate or ethanol precipitation, centrifugation, acid or base extraction, column chromatography, affinity column chromatography, anion or cation exchange chromatography, phosphocellulose chromatography, high performance liquid chromatography (HPLC), gel filtration, hydrophobic interaction chromatography, hydroxylapatite chromatography, lectin chromatography, gel electrophoresis and the like.

In addition to other references noted herein, a variety of protein purification methods are well known in the art, including, e.g., those set forth in R. Scopes, Protein Purification, Springer-Verlag, N.Y. (1982); Deutscher, Methods in Enzymology Vol. 182: Guide to Protein Purification, Academic Press, Inc. N.Y. (1990); Sandana (1997) Bioseparation of Proteins, Academic Press, Inc.; Bollag et al. (1996) Protein Methods, 2nd Edition Wiley-Liss, NY; Walker (1996) The Protein Protocols Handbook Humana Press, NJ; Harris and Angal (1990) Protein Purification Applications: A Practical Approach IRL Press at Oxford, Oxford, England; Harris and Angal Protein Purification Methods: A Practical Approach IRL Press at Oxford, Oxford, England; Scopes (1993) Protein Purification: Principles and Practice 3rd Edition Springer Verlag, NY; Janson and Ryden (1998) Protein Purification: Principles, High Resolution Methods and Applications, Second Edition Wiley-VCH, NY; and Walker (1998) Protein Protocols on CD-ROM Humana Press, NJ; and the references cited therein.

Well known techniques for refolding proteins can be used if necessary to obtain the active conformation of the protein when the protein is denatured during intracellular synthesis, isolation or purification. Methods of reducing, denaturing and renaturing proteins are well known to those of skill in the art (see the references above and Debinski, et al. (1993) J. Biol. Chem., 268: 14065-14070; Kreitman and Pastan (1993) Bioconjug. Chem.,4: 581-585; and Buchner, et al. (1992) Anal. Biochem., 205: 263-270).

The nucleotide sequence encoding the polypeptide can optionally be fused in-frame to a sequence encoding a module (e.g., a domain or tag) that facilitates purification of the polypeptide and/or facilitates association of the fusion polypeptide with a particle, a solid support or another reagent. Such modules include, but are not limited to, metal chelating peptides such as histidine-tryptophan modules that allow purification on and/or binding to immobilized metals (e.g., a hexahistidine tag), a sequence which binds glutathione (e.g., GST), a hemagglutinin (HA) tag (corresponding to an epitope derived from the influenza hemagglutinin protein; Wilson, I., et al. (1984) Cell 37:767), maltose binding protein sequences, the FLAG epitope utilized in the FLAGS extension/affinity purification system (Immunex Corp, Seattle, Wash.), and the like. The inclusion of a protease-cleavable polypeptide linker sequence between the purification domain and the sequence of the invention is useful to facilitate purification.

Any polypeptide can optionally be labeled, biotinylated or coupled with another moiety, either during or after synthesis. For example, a polypeptide can be fluorescently labeled using a commercially available kit, e.g., from Molecular Probes, Inc. (www.molecularprobes.com) or Pierce Biotechnology (www.piercenet.com). Similarly, a polypeptide can be biotinylated using commercially available kits or reagents, e.g., from Pierce Biotechnology (www.piercenet.com).

Production and Labeling of Antibodies

For the production of antibodies to a particular protein (e.g., for use as a capture and/or detection reagent for that protein), various host animals may be immunized by injection with the polypeptide or a portion thereof. Such host animals include, but are not limited to, rabbits, mice and rats, to name but a few. Various adjuvants may be used to enhance the immunological response, depending on the host species; adjuvants include, but are not limited to, Freund's (complete and incomplete), mineral gels such as aluminum hydroxide, surface active substances such as lysolecithin, pluronic polyols, polyanions, peptides, oil emulsions, keyhole limpet hemocyanin, dinitrophenol, and potentially useful human adjuvants such as BCG (bacille Calmette-Guerin) and Corynebacterium parvum.

Polyclonal antibodies are heterogeneous populations of antibody molecules derived from the sera of animals immunized with an antigen, such as a protein or an antigenic functional derivative thereof. For the production of polyclonal antibodies, host animals, such as those described above, may be immunized by injection with the protein, or a portion thereof, supplemented with adjuvants as also described above. The protein can optionally be produced and purified as described herein. For example, recombinant protein can be produced in a host cell, or a synthetic peptide derived from the sequence of the protein can be conjugated to a carrier protein and used as an immunogen. Standard immunization protocols are described in, e.g., Harlow and Lane (1988) Antibodies, A Laboratory Manual, Cold Spring Harbor Publications, New York. Additional references and discussion of antibodies is also found herein.

Monoclonal antibodies (mAbs), which are homogeneous populations of antibodies to a particular antigen, may be obtained by any technique which provides for the production of antibody molecules by continuous cell lines in culture. These include, but are not limited to, the hybridoma technique of Kohler and Milstein (Nature 256:495-497, 1975; and U.S. Pat. No. 4,376,110), the human B-cell hybridoma technique (Kosbor et al. (1983) Immunology Today 4:72; Cole et al. (1983) Proc. Natl. Acad. Sci. USA 80:2026-2030), and the EBV-hybridoma technique (Cole et al. (1985) Monoclonal Antibodies and Cancer Therapy, Alan R. Liss, Inc., pp. 77-96). Such antibodies may be of any immunoglobulin class, including IgG, IgM, IgE, IgA, IgD, and any subclass thereof. The hybridoma producing the mAb of this invention may be cultivated in vitro or in vivo.

In addition, techniques developed for the production of “chimeric antibodies” (Morrison et al. (1984) Proc. Natl. Acad. Sci. USA 81:6851-6855; Neuberger et al. (1984) Nature 312:604-608; Takeda et al. (1985) Nature 314:452-454) by splicing the genes from a mouse antibody molecule of appropriate antigen specificity together with genes from a human antibody molecule of appropriate biological activity, can be used. A chimeric antibody is a molecule in which different portions are derived from different animal species, such as those having a variable or hypervariable region derived from a murine mAb and a human immunoglobulin constant region.

Similarly, techniques useful for the production of “humanized antibodies” can be adapted to produce antibodies to the proteins, fragments or derivatives thereof. Such techniques are disclosed in U.S. Pat. Nos. 5,932,448; 5,693,762; 5,693,761; 5,585,089; 5,530,101; 5,569,825; 5,625,126; 5,633,425; 5,789,650; 5,661,016; and 5,770,429.

In addition, techniques described for the production of single-chain antibodies (U.S. Pat. No. 4,946,778; Bird (1988) Science 242:423-426; Huston et al. (1988) Proc. Natl. Acad. Sci. USA 85:5879-5883; and Ward et al. (1989) Nature 334:544-546) can be used. Single chain antibodies are formed by linking the heavy and light chain fragments of the Fv region via an amino acid bridge, resulting in a single-chain polypeptide.

Antibody fragments which recognize specific epitopes may be generated by known techniques. For example, such fragments include, but are not limited to, the F(ab′)₂ fragments, which can be produced by pepsin digestion of the antibody molecule, and the Fab fragments, which can be generated by reducing the disulfide bridges of the F(ab′)₂ fragments. Alternatively, Fab expression libraries may be constructed (Huse et al. (1989) Science 246:1275-1281) to allow rapid and easy identification of monoclonal Fab fragments with the desired specificity.

A large number of antibodies are commercially available. For example, monoclonal and/or polyclonal antibodies against any of a large number of specific proteins, against phosphoserine, against phosphothreonine, against phosphotyrosine, and against any phosphoprotein (i.e., against phosphoserine, phosphothreonine and phosphotyrosine) are available, for example, from Zymed Laboratories, Inc. (www.zymed.com), QIAGEN, Inc. (www.qiagen.com) and BD Biosciences (www.bd.com), among many other sources. In addition, a number of companies offer services that produce antibodies against the desired antigen (e.g., a protein supplied by the customer or a peptide synthesized to order), including Abgent (www.abgent.com), QIAGEN, Inc. (www.merlincustomservices.com) and Zymed Laboratories, Inc. (www.zymed.com).

Optionally, a fluorescent label (e.g., a fluorophore such as fluorescein, Alexa Fluor 488, phycoerythrin or rhodamine) can be chemically coupled to antibodies without altering their binding capacity (e.g., by use of a commercially available kit for labeling antibodies, such as the kits available from Molecular Probes, Inc. (www.molecularprobes.com) and Pierce Biotechnology (www.piercenet.com)). When activated by illumination with light of a particular wavelength, the fluorescent label on the antibody absorbs the light energy, inducing a state of excitability in the molecule, followed by emission of the light at a characteristic longer wavelength. The emission appears as a characteristic color visually detectable with a light microscope, flow cytometer or other suitable instrument. Such techniques are very well established in the art. Similarly, other moieties such as enzymes, gold particles, biotin, etc. can be coupled to antibodies. For example, kits and reagents for biotinylating antibodies (e.g., for subsequent detection of the biotinylated antibody with fluorescently labeled avidin or streptavidin) are commercially available, e.g., from Pierce Biotechnology (www.piercenet.com). Alternatively, one or more antibodies of a given species can be detected with a labeled anti-species antibody (e.g., mouse antibodies can be detected with a goat anti-mouse antibody).

Microspheres

Microspheres are preferred particles for the practice of this invention, since they are generally stable, are widely available in a range of materials, surface chemistries and uniform sizes, and can be fluorescently dyed. Microspheres can be distinguished from each other by identifying characteristics such as their size (diameter) and/or their fluorescent emission spectra.

Luminex Corp. (www.luminexcorp.com), for example, offers 100 sets of uniform diameter polystyrene microspheres. The microspheres of each set are internally labeled with a distinct ratio of two fluorophores. A flow cytometer or other suitable instrument can thus be used to classify each individual microsphere according to its predefined fluorescent emission ratio. Fluorescently-coded microsphere sets are also available from a number of other suppliers, including Radix Biosolutions (www.radixbiosolutions.com) and Upstate Biotechnology (www.upstatebiotech.com). Alternatively, BD Biosciences (www.bd.com) and Bangs Laboratories, Inc. (www.bangslabs.com) offer microsphere sets distinguishable by a combination of fluorescence and size. As another example, microspheres can be distinguished on the basis of size alone, but fewer sets of such microspheres can be multiplexed in an assay because aggregates of smaller microspheres can be difficult to distinguish from larger microspheres.

Microspheres with a variety of surface chemistries are commercially available, from the above suppliers and others (e.g., see additional suppliers listed in Kellar and lannone (2002) “Multiplexed microsphere-based flow cytometric assays” Experimental Hematology 30:1227-1237 and Fitzgerald (2001) “Assays by the score” The Scientist 15[11]:25). For example, microspheres with carboxyl, hydrazide or maleimide groups are available and permit covalent coupling of molecules (e.g., capture reagents such as polypeptides, nucleic acids, carbohydrates, or other molecules with free amine, carboxyl, aldehyde, sulfhydryl or other reactive groups) to the microspheres. Microspheres with surface avidin or streptavidin are available and can bind biotinylated capture reagents; similarly, microspheres coated with biotin are available for binding capture reagents conjugated to avidin or streptavidin. Microspheres coated with anti-species antibodies (e.g., with anti-mouse IgG), protein A and protein G are available for binding antibody capture reagents. Microspheres coated with Ni²⁺ or glutathione are available and permit binding of polyhistidine-tagged or GST-tagged recombinant polypeptides, respectively. In addition, services that couple a capture reagent of the customer's choice to microspheres are commercially available, e.g., from Radix Biosolutions (www.radixbiosolutions.com).

Protocols for using such commercially available microspheres (e.g., methods of covalently coupling proteins and nucleic acids to carboxylated microspheres, methods of blocking reactive sites on the microsphere surface that are not occupied by the capture reagents, methods of binding biotinylated capture reagents to avidin-functionalized microspheres, and the like) are typically supplied with the microspheres and are readily utilized and/or adapted by one of skill. In addition, coupling of capture reagents to microspheres is well described in the literature. For example, see Fulton et al. (1997) “Advanced multiplexed analysis with the FlowMetrix™ system” Clinical Chemistry 43:1749-1756; Jones et al. (2002) “Multiplex assay for detection of strain-specific antibodies against the two variable regions of the G protein of respiratory syncytial virus” 9:633-638; Camilla et al. (2001) “Flow cytometric microsphere-based immunoassay: Analysis of secreted cytokines in whole-blood samples from asthmatics” Clinical and Diagnostic Laboratory Immunology 8:776-784; Martins (2002) “Development of internal controls for the Luminex instrument as part of a multiplexed seven-analyte viral respiratory antibody profile” Clinical and Diagnostic Laboratory Immunology 9:41-45; Kellar and Iannone (2002) “Multiplexed microsphere-based flow cytometric assays” Experimental Hematology 30:1227-1237; Oliver et al. (1998) “Multiplexed analysis of human cytokines by use of the FlowMetrix system” Clinical Chemistry 44:2057-2060; Gordon and McDade (1997) “Multiplexed quantification of human IgG, IgA, and IgM with the FlowMetrix™ system” Clinical Chemistry 43:1799-1801; U.S. Pat. No. 5,981,180 entitled “Multiplexed analysis of clinical specimens apparatus and methods” to Chandler et al. (Nov. 9, 1999); U.S. Pat. No. 6,449,562 entitled “Multiplexed analysis of clinical specimens apparatus and methods” to Chandler et al. (Sep. 10, 2002); and references therein.

Methods of binding proteins or other macromolecules to capture reagents coupled to microspheres are also described in the above references, as are methods for producing and using detection reagents. Methods of analyzing microsphere populations (e.g., methods of identifying microsphere subsets by their size and/or fluorescence characteristics, methods of using size to distinguish microsphere aggregates from single uniformly sized microspheres and eliminate aggregates from the analysis, methods of detecting the presence or absence of a detection reagent on the microsphere subset, and the like) are also well described in the literature. See, e.g., the above references.

Suitable instruments, software, and the like for analyzing microsphere populations to distinguish subsets of microspheres and to detect the presence or absence of detection reagents on each subset are commercially available. For example, flow cytometers are widely available, e.g., from Becton-Dickinson (www.bd.com) and Beckman Coulter (www.beckman.com). Luminex 100™ and Luminex HTS™ systems (which use microfluidics to align the microspheres and two lasers to excite the microspheres and the label for the detection reagent) are available from Luminex Corp. (www.luminexcorp.com); the similar Bio-Plex™ Protein Array System is available from Bio-Rad Laboratories, Inc. (www.bio-rad.com). A confocal microplate reader suitable for microsphere analysis, the FMAT™ System 8100, is available from Applied Biosystems (www.appliedbiosystems.com).

Arrays

In an array of capture reagents on a solid support (e.g., a membrane or a glass or plastic slide or plate), each capture reagent is bound (e.g., electrostatically or covalently bound, directly or via a linker) to the support at a unique location. Methods of making, using, and analyzing such arrays (e.g., microarrays) are well known in the art. See, e.g., U.S. Pat. No. 6,197,599; MacBeath and Schreiber (2000) “Printing proteins as microarrays for high-throughput function determination” Science 289:1760-1763 and the accompanying web site http://cgr.harvard.edu/macbeath/protocols/proteinmicroarrays.html; Ziauddin and Sabatini (2001) “Microarrays of cells expressing defined cDNAs” Nature 411(6833):107-10; Falsey et al. Bioconjug. Chem. (2001) 12:346-53; Reimer et al. (2002) Curr. Opin. Biotech. 13:315-320; Huang (2001) J. Immunological Methods 255:1-13; Kim et al. (2002) “Quantitative measurement of serum allergen-specific IgE on protein chip” Experimental and Molecular Medicine 34:152-158; and Mezzasoma et al. (2002) “Antigen microarrays for serodiagnosis of infectious diseases” Clin. Chem. 48:121-130. Arrays can be formed (e.g., printed), for example, using commercially available instruments such as a GMS 417 Arrayer (Affymetrix, Santa Clara, Calif.).

Suitable solid supports are commercially readily available. For example, a variety of membranes (e.g., nylon, PVDF, and nitrocellulose membranes) are commercially available, e.g., from Sigma-Aldrich, Inc. (www.sigmaaldrich.com). As another example, surface-modified and pre-coated slides with a variety of surface chemistries are commercially available, e.g., from TeleChem International (www.arrayit.com), Corning, Inc. (Corning, N.Y.), or Greiner Bio-One, Inc. (www.greinerbiooneinc.com). For example, silanated and silyated slides with free amino and aldehyde groups, respectively, are available and permit covalent coupling of molecules (e.g., capture reagents such as polypeptides, nucleic acids, carbohydrates, or other molecules with free aldehyde, amine, or other reactive groups) to the slides. Slides with surface streptavidin are available and can bind biotinylated capture reagents; similarly, slides coated with Ni 2+are available and permit binding of polyhistidine-tagged recombinant polypeptides. In addition, services that produce arrays of peptides or nucleic acids of the customer's choice are cominmercially available, e.g., from TeleChem International (www.arrayit.com).

### EXAMPLES

The following sets forth a series of experiments that demonstrate multiplex detection of a phosphorylated tyrosine residue in a plurality of proteins using a single detection reagent. It is understood that the examples and embodiments described herein are for illustrative purposes only and that various modifications or changes in light thereof will be suggested to persons skilled in the art and are to be included within the spirit and purview of this application and scope of the appended claims. Accordingly, the following examples are offered to illustrate, but not to limit, the claimed invention.

Materials and Methods

I. Cells and Cell Lines

Jurkat T-cell line (clone E6-1) was obtained from the American Type Culture Collection (ATCC). Jurkat cells were grown as a suspension in RPMI media containing 10% fetal bovine serum (FBS). A431 cervical carcinoma cell line was also obtained from the ATCC and maintained as adherent monolayers in Delbecco's Modified Eagle's Media (DMEM) containing 10% FBS.

II. Antibodies

Monoclonal antibodies against signaling proteins Lck (clone 3A5), Zap70 (clone 2F3.2), LAT (clone 2E9) and EGFR (clone LA22) were purchased from Upstate Inc. (Lake Placid, N.Y.) and used as capture reagents. Monoclonal antibody against phosphotyrosine (4G10, biotinylated) was also purchased from Upstate Inc. and used as the detection reagent.

III. Multiplex System

Luminex microspheres (beads) and the Luminex-100™ instrument were manufactured by Luminex Corp. (Austin, Tex.). Up to a hundred distinct microsphere sets are available from Luminex Corp. that are identifiable by a unique fluorescent signature for each microsphere set. The fluorescent signature can be detected, e.g., by the Luminex-100™ instrument. The identity of each microsphere set in combination with the known capture reagent (or control protein or the like) coated onto a particular microsphere set provides the basis for the multiplexing capabilities of the Luminex detection system.

IV. Coupling Proteins to Luminex Microspheres

Luminex microspheres were coated with various proteins, either as capture reagents or as controls, by chemical cross-linking according to the manufacturer's instructions, as follows. For multiplex experiments, each subset of microspheres was covalently coupled to a different protein.

For each set (subset) of microspheres, microsphere stock was resuspended by vortexing and sonication (15 to 30 sec.). An aliquot of 2×10⁶ microspheres was removed and centrifuged at 12,000×g for 2 min. Microspheres were resuspended in 801 μl of activation buffer (100 mM monobasic sodium phosphate; pH 6.3) by vortexing and sonication (15 to 30 sec.).

To prepare the microspheres for cross-linking to proteins, 10 μl of 50 mg/ml Sulfo-NHS(N-hydroxysulfosuccinamide; Pierce, Rockford, Ill.) was added and microspheres were mixed by vortexing. Then 10 μl of 50 mg/ml EDC (1-ethyl-3-[3-dimethylaminopropyl] carbodiimide; Pierce, Rockford, Ill.) was added and microspheres were mixed again by vortexing. The microsphere mixture was incubated shaking on a rocker at room temperature (RT) for 20 min and centrifuged at 12,000×g for 2 min. Microspheres were washed twice with 1 ml of 50 mM MES (pH 6.0) buffer.

Each set (subset) of microspheres was coated with one antibody or control protein. To coat each set of microspheres with a particular protein, pelleted microspheres were resuspended in the relevant protein solution, prepared as follows. Monoclonal antibodies against various signaling proteins were diluted in 50 mM MES (pH 6.0) buffer to the final concentration listed: 25 μg/ml anti-Lck antibody, 50 μg/ml anti-Zap70 antibody, 25 μg/ml anti-LAT antibody, and 100 μg/ml anti-EGFR antibody. A control antibody (biotin conjugated Goat IgG) and a control protein (bovine serum albumin (BSA)) were also each diluted in 50 mM MES (pH 6.0) buffer to a final concentration of 100 μg/ml.

Each mixture of activated microspheres and protein was incubated shaking on a rocker for 2 hr at RT for coupling. After coating with protein, microspheres were washed twice with wash buffer (0.1% Tween-20 in phosphate buffered saline (PBS), pH 7.4) and resuspended in 1 ml of blocking buffer (1% BSA; 0.1% Tween-20 in PBS, pH 7.4; 0.05% sodium azide). Blocking was performed by shaking on a rocker at RT for 30 min. After blocking, microspheres were washed twice in 1 ml blocking buffer. Finally, the protein-coupled microspheres were resuspended in 1 ml blocking buffer and stored at 4° C. for up to a week. For long-term storage, the protein-coupled microspheres were kept frozen at −70° C. for several months. The protein-coated microspheres were more stable at −70° C. than at 4° C. No additional cryoprotectant (e.g., DMSO, glycerol or the like) was required for storage at −70° C., and repeated freeze-thaw cycles did not affect the performance of the coated microspheres in the assay.

V. Cell Activation and Lysis

Cells were activated so that signaling proteins would become tyrosine-phosphorylated. Cells were activated by a general, non-specific activation procedure as well as by pathway specific procedures.

General Cell Activation

Jurkat cells and A431 cells were treated with sodium pervanadate, which inhibits intracellular tyrosine phosphatases. This treatment results in hyperphosphorylation of tyrosine kinases and their substrates. Sodium pervanadate was prepared by adding 330 μl of 30% hydrogen peroxide to 1 ml of 20 mM sodium vanadate (pH 10.0). The mixture was incubated at RT for 15 min before addition to cells.

Jurkat cells were resuspended to a density of 1×10⁸ cells/ml in PBS. Cells were pre-warmed to 37° C. for 15 min. To each milliliter of cell suspension, 40 μl of sodium pervanadate was added. Cells were mixed and immediately incubated at 37° C. for 5 min. Activated cells were lysed, and cell lysates were processed as described below.

A431 cells were grown to 80-90% confluency in T75 flasks. Cells were washed once with fresh media (37° C.). For treatment, sodium pervanadate was prepared as described above and diluted into DMEM to 1 mM final concentration. Two ml of 1 mM sodium pervanadate containing media, prewarmed to 37° C., was added per T75 flask and the cell monolayer was covered with it. Cells were incubated at 37° C. for 5 min. Activated cells were lysed and processed as described below.

Pathway Specific Cell Activation

For pathway specific activation, Jurkat cells were treated with anti-CD3-receptor monoclonal antibody (UCHT1) (Becton Dickenson, San Diego, Calif.). Cells were resuspended in PBS at a density of 1×10⁸/ml and prewarmed to 37° C. Anti-CD3 antibody was added to a final concentration of 5 μg/ml. Cells were mixed and incubated at 37° C. for various times ranging from 5 sec to 10 min. Optimal phosphorylation of most tyrosine kinases and their substrates in the T-cell receptor pathway occurred in 5 min.

For pathway specific activation of A431 cells, which overexpress the epidermal growth factor receptor (EGFR), the cells were treated with epidermal growth factor (EGF). Cells were grown to about 80-90% confluency in a T75 flask. The optimal concentration of EGF for activation of the EGFR pathway in A431 cells was determined to be 100 μM. EGF was diluted in DMEM containing 10% serum. Two ml of the diluted EGF (100 μM) was layered on top of the cell monolayer. Cells were incubated at 37° C. for 15 min for activation. Activated cells were lysed and processed as described below.

Preparation of Lysate from Activated Cells

Jurkat cells were lysed by adding a volume of 2× lysis buffer (2% NP-40 and protease inhibitor cocktail (both from Roche Diagnostics, Mannheim, Germany) in PBS), pre-chilled on ice, equal to the volume of the resuspended cells. Lysate was vortexed immediately after addition of the lysis buffer and incubated on ice for 15 min. While incubating on ice, it was mixed by vortexing every 5 min.

A431 cells were lysed by adding 1 ml of 1× lysis buffer (1% NP-40 and protease inhibitor cocktail in PBS), pre-chilled on ice, per T75 flask. Cells were removed by scraping with a cell scraper. Lysate was vortexed immediately after addition of lysis buffer and incubated on ice for 15 min. While incubating on ice, it was mixed by vortexing every 5 min.

After the 15 min incubation to complete cell lysis, the lysate was centrifuged at 15,000×g at 4° C. for 30 min to remove cell debris. Clear lysate was either used immediately for assay of tyrosine-phosphorylated signaling proteins or stored frozen at −70° C. until use.

VI. Multiplex Immunoassay Using the Luminex System

Immunoreactions were set up in 96 well, filter bottomed plates designed for high throughput separations (1.2 μm MultiScreen, Millipore Corporation, Bedford, Mass.). Typically, up to 1000 microspheres for each individual microsphere set, coated with a known protein (antibody or control protein), were added per well. For example, for a five-plex assay, 1000 microspheres from each set coated with a known protein (e.g., a known antibody) were mixed to provide a total of 5,000 microspheres. This five-plex microsphere mixture was added per well in a total of 50 μl blocking buffer. To this, 50 μl of cell lysate (representing 5×10⁶ Jurkat cells) was added and mixed by pipetting up and down three to four times. The mixture of microspheres and lysate was then incubated on a shaker for 2 hr at RT.

After incubation with the lysate, liquid was drained from the bottom of plate, under vacuum, on a suction apparatus (Millipore Corporation) designed to fit these plates. The microspheres were washed three times by adding 150 μl of wash buffer per well and draining out under vacuum successively. For detection of phosphorylated cellular proteins, bound to specific antibodies conjugated to microspheres, 50 μl of biotinylated anti-phosphotyrosine antibody 4G10 (1:1000 dilution in wash buffer) was added as the detection reagent. Microspheres were mixed as before and incubated at RT for 30 min. Following incubation with 4G10-biotin, microspheres were washed three times as before. To detect biotinylated 4G10, streptavidin conjugated to R-phycoerythrin (CalTag, Burlingame, Calif.) was added at a dilution of 1:500 in wash buffer as the secondary agent. The contents of each well were mixed and incubated at RT for 15 min. Microspheres were washed three times with wash buffer. Washed microspheres were resuspended in 100 μl of wash buffer per well and analyzed in the Luminex-100™ instrument, according to the manufacturer's instructions.

The Luminex-100™ instrument was used at the default settings recommended by the manufacturer for routine use, as directed by the User's Manual accompanying the instrument. The instrument was supplied with a complete software package for operation of the instrument, called Luminex Data Collection Software. This software allowed routine operation, data acquisition, and data analysis. Version 1.7 of the software was used according to instructions in the User's Manual supplied by the manufacturer. After initial data analysis using the Luminex software, data were plotted using Microsoft Excel.

Results

I. Analysis of Tyrosine-Phosphorylation of EGFR in Activated A431 Cells

Initial experiments using microspheres coated with anti-EGFR antibodies confirmed that tyrosine phosphorylated EGFR could be detected in lysates of pervanadate-activated and EGF-activated A431 cells, using the biotinylated anti-phosphotyrosine antibody 4G10 as the detection reagent, R-phycoerythrin-labeled streptavidin as the secondary agent, a microsphere subset coated with anti-EGFR antibody as the capture reagent, and the Luminex system. Later experiments focused on analyzing multiple proteins in another pathway simultaneously, as described below. (It will be evident that multiple proteins in the EGFR pathway or any other pathways or combinations thereof could similarly be analyzed simultaneously.)

II. Multiplex Analysis of Tyrosine-Phosphorylation of Lck, Zap70, and LAT in Jurkat T-Cells Activated by Sodium Pervanadate

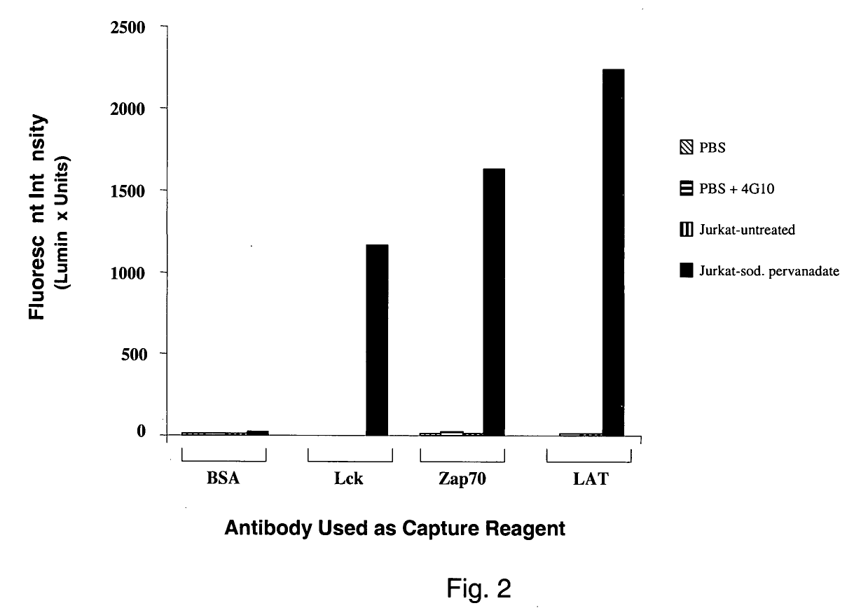

Pervanadate treatment of cells resulted in inhibition of tyrosine phosphatases. This gave rise to hyperphosphorylation of tyrosine kinases and their substrates. Thus, lysate prepared from Jurkat T-cells treated with sodium pervanadate provided for signaling proteins heavily phosphorylated on tyrosine residues. A five-plex assay was performed to detect tyrosine phosphorylation, as follows. Three subsets of Luminex microspheres coated with monoclonal antibodies to signaling proteins Lck, Zap70, or LAT and two subsets coated with control proteins IgG-biotin or BSA were mixed and incubated with cell lysates as described above. FIG. 2 shows that phosphorylation specific signal was detected in the assay. Microspheres coated with capture reagents (antibodies to Lck, Zap70 and LAT) showed a strong fluorescent signal from streptavidin-phycoerythrin after reaction with lysate from pervanadate treated Jurkat cells (FIG. 2, black bars). Lysate from untreated cells (vertically hatched bars) yielded a signal similar to negative control samples such as PBS alone (cross hatched bars) and PBS followed by incubation with biotinylated 4G10 (horizontally hatched bars). Similarly, only background signal was detected in microspheres coated with BSA in lysates from both untreated and pervanadate treated Jurkat cells.

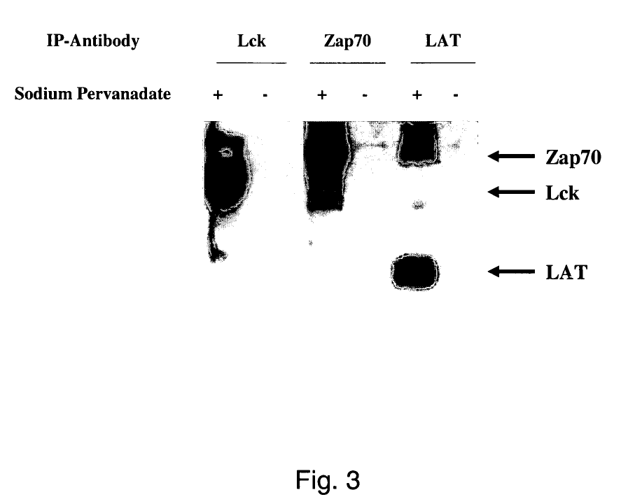

To confirm that microspheres coated with antibodies to Lck, Zap70 and LAT captured predominantly specific proteins in cell lysates, immunoprecipitation was performed with these antibodies. Cell lysates (1×10⁸ cells in 1 ml) were incubated with 2.5 μg of each antibody for 1 hr. The immune complexes were captured with Protein G conjugated sepharose (Sigma Chemicals, St. Louis, Mo.) and resolved by standard polyacrylamide gel electrophoresis (PAGE). Resolved proteins were transferred to PVDF membrane (BiORad, Hercules, Calif.) and probed with biotinylated 4G10 (1:2000 dilution in 5% non-fat powdered milk in PBS). Phosphorylated bands were finally detected by ABC Detection Reagent (Vector Laboratories, Burlingame, Calif.) and Chemiluminescent Substrate (Pierce). FIG. 3 shows that phosphorylated bands of Lck, Zap70, and LAT (arrows) were detected only in lysates from cells treated with sodium pervanadate, and only in immunoprecipitates with the relevant antibodies. These phosphorylated proteins predominated in their respective immunoprecipitates.

Multiplex analysis of Lck, Zap70 and LAT was also performed in pervanadate treated peripheral blood mononuclear cells (which are primary cells, rather than a cell line), with similar results.

The above data clearly show the utility of the multiplex microsphere suspension assay approach to analyzing the phosphorylation state of multiple signaling proteins in a single sample with a single detection reagent (i.e., separate detection reagents, one to detect phosphorylation of each protein, are not required).

III. Multiplex Analysis of Tyrosine-Phosphorylation of Lck, Zap70, and LAT in Jurkat T-Cells Activated by Anti-CD3 Antibody

For pathway specific activation of signaling proteins, Jurkat cells were treated with anti-CD3 antibody (UCHT1) as described above for various times between 5 sec and 10 min. Such treatment is known to result in CD3 receptor stimulation, giving rise to a physiologically relevant activation of T-cells. This stimulation results in rapid phosphorylation of tyrosine kinase Lck. Lck in turn phosphorylates the tyrosine kinase Zap70. Once activated, Zap70 phosphorylates its direct substrate, an adapter molecule LAT.

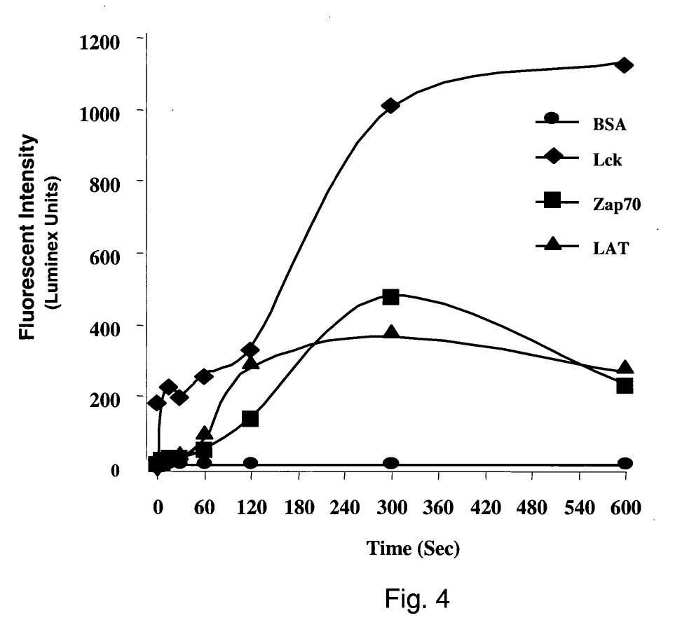

A five-plex mixture of microspheres coated with antibodies to Lck, Zap70 and LAT and control proteins BSA and biotinylated IgG, as above, was reacted with cell lysates from anti-CD3 treated and untreated Jurkat cells. FIG. 4 shows a time-course of activation of the three signaling proteins. Within 5 sec of addition of anti-CD3 antibody, Lck (diamonds) was phosphorylated; phosphorylation peaked at 5 min. This was followed by phosphorylation of Zap70 (squares) and LAT (triangles) after 2 min. Phosphorylation of these two peaked at 5 min also. As shown, microspheres coated with BSA (circles) displayed only a background signal. Similarly, lysate from untreated Jurkat cells resulted only in a background signal with Lck, Zap70 and LAT coated microspheres, similar to those coated with BSA.

While the foregoing invention has been described in some detail for purposes of clarity and understanding, it will be clear to one skilled in the art from a reading of this disclosure that various changes in form and detail can be made without departing from the true scope of the invention. For example, all the techniques and apparatus described above can be used in various combinations. All publications, patents, patent applications, and/or other documents cited in this application are incorporated by reference in their entirety for all purposes to the same extent as if each individual publication, patent, patent application, and/or other document were individually indicated to be incorporated by reference for all purposes.

1. A method of detecting the presence or absence of a first posttranslational modification of a plurality of proteins in a sample, the method comprising:
providing the sample comprising the proteins;
providing a pooled population of a plurality of subsets of particles, the particles in each subset comprising a capture reagent specific for at least one of the proteins, and the particles in each subset being distinguishable from those of every other subset;
providing a single first detection reagent, the first detection reagent providing an indication of the presence of the first posttranslational modification;
binding the proteins to the capture reagents;
exposing the proteins to the first detection reagent; and,
determining whether each of the proteins comprises the first posttranslational modification by identifying each subset of particles and detecting the presence or absence of the first detection reagent on each subset of particles.

2. The method of claim 1, wherein the particles in each subset comprise a capture reagent specific for one of the proteins.

3. The method of claim 1, wherein binding the proteins to the capture reagents comprises exposing the pooled population of subsets of particles to the sample, and wherein exposing the proteins to the first detection reagent comprises adding the first detection reagent to the exposed pooled population.

4. The method of claim 3, comprising washing the exposed pooled population prior to adding the first detection reagent.

5. The method of claim 1, wherein the first posttranslational modification is phosphorylation.

6. The method of claim 5, wherein the first posttranslational modification is phosphorylation of a serine, threonine or tyrosine residue, or a combination thereof.

7. The method of claim 1, wherein the first posttranslational modification is ubiquitination, sumoylation, glycosylation, prenylation, myristoylation, farnesylation or acetylation.

8. The method of claim 1, wherein the particles are microspheres.

9. The method of claim 8, wherein the microspheres of each subset are distinguishable from those of the other subsets on the basis of their fluorescent emission spectra, their diameter, or a combination thereof.

10. The method of claim 1, wherein the capture reagent comprises one or more of: a nucleic acid, an oligonucleotide, a polypeptide, an antibody, a recombinant protein, a synthetic peptide, a substrate analog or a ligand.

11. The method of claim 1, wherein the first detection reagent comprises one or more of: a nucleic acid, an oligonucleotide, a polypeptide, an antibody, a recombinant protein, or a synthetic peptide.

12. The method of claim 1, wherein the first detection reagent is an antibody specific for a phosphorylated tyrosine, serine or threonine residue, or a combination thereof.

13. The method of claim 1, wherein the first detection reagent is an antibody specific for ubiquitin, an antibody specific for a carbohydrate moiety or an antibody specific for an acetyl group.

14. The method of claim 1, wherein the first detection reagent comprises a first fluorescent label, and wherein detecting the presence or absence of the first detection reagent comprises detecting a first fluorescent signal from the first label.

15. The method of claim 1, wherein detecting the presence or absence of the first detection reagent comprises: adding a labeled secondary agent that binds the first detection reagent and detecting a signal from the labeled secondary agent.

16. The method of claim 1, wherein the proteins comprise one or more of: an endogenous cellular protein or a protein encoded by an infectious agent.

17. The method of claim 1, wherein the plurality of proteins comprises a plurality of protein kinases.

18. The method of claim 1, wherein the sample is derived from an animal, a human, a plant, a cultured cell or a microorganism.

19. The method of claim 1, wherein the sample comprises one or more of: a cell lysate, an intercellular fluid, a conditioned culture medium or a bodily fluid.

20. The method of claim 1, wherein the sample is derived from a tissue, a biopsy or a tumor.

21. The method of claim 1, comprising recovering at least one of the subsets of particles.

22. The method of claim 1, comprising:
providing a second detection reagent;
exposing the proteins to the second detection reagent; and,
detecting the presence or absence of the second detection reagent on each subset of particles.

23. The method of claim 22, wherein the second detection reagent provides an indication of the presence of a second posttranslational modification.

24. A method of diagnosing or monitoring disease by detecting the presence or absence of a phosphorylated amino acid residue in a plurality of protein kinases, the method comprising:
providing a sample comprising the protein kinases;
providing a pooled population of a plurality of subsets of particles, the particles in each subset comprising a capture reagent specific for at least one of the kinases, and the particles in each subset being distinguishable from those of every other subset;
providing a single first detection reagent, the first detection reagent providing an indication of the presence of the phosphorylated amino acid residue;
binding the protein kinases to the capture reagents;
exposing the protein kinases to the first detection reagent;
generating a kinase activity profile for the sample by determining whether each of the kinases comprises the phosphorylated amino acid residue by identifying each subset of particles and detecting the presence or absence of the first detection reagent on each subset of particles; and,
comparing the kinase activity profile for the sample with one or more control kinase activity profiles.

25. The method of claim 24, wherein the particles in each subset comprise a capture reagent specific for one of the kinases.

26. The method of claim 24, wherein binding the protein kinases to the capture reagents comprises exposing the pooled population of subsets of particles to the sample, and wherein exposing the protein kinases to the first detection reagent comprises adding the first detection reagent to the exposed pooled population.

27. The method of claim 26, comprising washing the exposed pooled population prior to adding the first detection reagent.

28. The method of claim 24, wherein the phosphorylated amino acid residue is a serine, threonine or tyrosine residue, or a combination thereof.

29. The method of claim 24, wherein the particles are microspheres.

30. The method of claim 29, wherein the microspheres of each subset are distinguishable from those of the other subsets on the basis of their fluorescent emission spectra, their diameter, or a combination thereof.

31. The method of claim 24, wherein the capture reagents are antibodies.

32. The method of claim 24, wherein the first detection reagent comprises one or more of: a nucleic acid, an oligonucleotide, a polypeptide, an antibody, a recombinant protein or a synthetic peptide.

33. The method of claim 24, wherein the first detection reagent is an antibody specific for a phosphorylated tyrosine, serine or threonine residue, or a combination thereof.

34. The method of claim 24, wherein the first detection reagent comprises a fluorescent label, and wherein detecting the presence or absence of the first detection reagent comprises detecting a fluorescent signal from the label.

35. The method of claim 24, wherein detecting the presence or absence of the first detection reagent comprises: adding a labeled secondary agent that binds the first detection reagent and detecting a signal from the labeled secondary agent.

36. The method of claim 24, wherein the kinases comprise one or more of: an endogenous cellular protein or a protein encoded by an infectious agent.

37. The method of claim 24, wherein the sample is derived from an animal, a human or a plant.

38. The method of claim 24, wherein the sample comprises a cell lysate.

39. The method of claim 24, wherein the sample is derived from a tissue, a biopsy or a tumor.

40. The method of claim 24, wherein the control kinase activity profiles comprise one or more of: a kinase activity profile for a normal, healthy cell; a kinase activity profile for a diseased cell; or a kinase activity profile for a second sample from the same source, taken at a different time.

41. The method of claim 24, comprising recovering at least one of the subsets of particles.

42. The method of claim 24, comprising:
providing a second detection reagent;
exposing the protein kinases to the second detection reagent; and,
detecting the presence or absence of the second detection reagent on each subset of particles.

43-63. (Cancelled).

64. A composition, comprising:
a plurality of subsets of particles, the particles in each subset comprising a capture reagent specific for at least one of a plurality of proteins comprising or suspected of comprising a first posttranslational modification, and the particles in each subset being distinguishable from those of every other subset; and,
a single first detection reagent, the first detection reagent providing an indication of the presence of the first posttranslational modification.

65. The composition of claim 64, wherein the particles in each subset comprise a capture reagent specific for one of the plurality of proteins.

66. The composition of claim 64, comprising the plurality of proteins comprising or suspected of comprising the first posttranslational modification.

67. The composition of claim 66, wherein each of the plurality of proteins is associated with one of the subsets of particles.

68. The composition of claim 66, wherein the proteins comprise one or more of: an endogenous cellular protein or a protein encoded by an infectious agent.

69. The composition of claim 66, wherein the plurality of proteins comprises a plurality of protein kinases.

70. The composition of claim 64, wherein the first posttranslational modification is phosphorylation of a serine, threonine or tyrosine residue, or a combination thereof.

71. The composition of claim 64, wherein the first posttranslational modification is ubiquitination, sumoylation, glycosylation, prenylation, myristoylation, farnesylation or acetylation.

72. The composition of claim 64, wherein the particles are microspheres.

73. The composition of claim 72, wherein the microspheres of each subset are distinguishable from those of the other subsets on the basis of their fluorescent emission spectra, their diameter, or a combination thereof.

74. The composition of claim 64, wherein the capture reagent comprises one or more of: a nucleic acid, an oligonucleotide, a polypeptide, an antibody, a recombinant protein, a synthetic peptide, a substrate analog or a ligand.

75. The composition of claim 64, wherein the first detection reagent comprises one or more of: a nucleic acid, an oligonucleotide, a polypeptide, an antibody, a recombinant protein, or a synthetic peptide.

76. The composition of claim 64, wherein the first detection reagent is an antibody specific for a phosphorylated tyrosine, serine or threonine residue, or a combination thereof.

77. The composition of claim 64, wherein the first detection reagent is an antibody specific for ubiquitin, an antibody specific for a carbohydrate moiety, or an antibody specific for an acetyl group.

78. The composition of claim 64, wherein the first detection reagent comprises a first fluorescent label.

79. The composition of claim 64, comprising a labeled secondary agent that binds the first detection reagent.

80. The composition of claim 64, comprising a second detection reagent.

81. The composition of claim 80, wherein the second detection reagent provides an indication of the presence of a second posttranslational modification.

82. A system comprising the composition of claim 64 and one or more fluid or particle handling or fluid or particle containing elements.

83. A kit comprising each of the components of the composition of claim 64 and instructions for using the composition to detect at least one posttranslational modification, packaged in one or more containers.

84-108. (Cancelled).

109. A kit for detecting the presence or absence of a first posttranslational modification of a plurality of proteins in a sample, comprising:
a plurality of subsets of particles, the particles in each subset being distinguishable from those of every other subset; and,
a single first detection reagent capable of providing an indication of the presence of the first posttranslational modification,
packaged in one or more containers.

110. The kit of claim 109, wherein the particles in each subset comprise a capture reagent specific for at least one of the proteins.

111. The kit of claim 110, wherein the capture reagent is specific for one of the proteins.

112. The kit of claim 110, wherein the proteins are protein kinases and wherein each capture reagent is specific for one of the protein kinases.

113. The kit of claim 110, wherein the capture reagent comprises one or more of a nucleic acid, an oligonucleotide, a polypeptide, an antibody, a recombinant protein, a synthetic peptide, a substrate analog or a ligand.

114. The kit of claim 109, wherein the first posttranslational modification is phosphorylation of a serine, threonine or tyrosine residue, or a combination thereof.

115. The kit of claim 109, wherein the first posttranslational modification is ubiquitination, sumoylation, glycosylation, prenylation, myristoylation, farnesylation or acetylation.

116. The kit of claim 109, wherein the particles are microspheres.

117. The kit of claim 116, wherein the microspheres of each subset are distinguishable from those of the other subsets on the basis of their fluorescent emission spectra, their diameter, or a combination thereof.

118. The kit of claim 109, wherein the first detection reagent comprises one or more of: a nucleic acid, an oligonucleotide, a polypeptide, an antibody, a recombinant protein, or a synthetic peptide.

119. The kit of claim 109, wherein the first detection reagent is an antibody specific for a phosphorylated tyrosine, serine or threonine residue, or a combination thereof.

120. The kit of claim 109, wherein the first detection reagent is an antibody specific for ubiquitin, an antibody specific for a carbohydrate moiety or an antibody specific for an acetyl group.

121. The kit of claim 109, wherein the first detection reagent comprises a fluorescent label.

122. The kit of claim 109, wherein the kit comprises a labeled secondary agent that binds the first detection reagent.

123. The kit of claim 109, comprising a second detection reagent.

124. The kit of claim 123, wherein the second detection reagent provides an indication of the presence of a second posttranslational modification.

125. The kit of claim 109, comprising instructions for use of the kit.

126. The kit of claim 125, wherein the instructions comprise: instructions for attaching a capture reagent to each subset of particles, if the capture reagent is not already attached; instructions for binding the proteins to the capture reagents; instructions for exposing the proteins to the first detection reagent; instructions for determining whether each of the proteins comprises the first posttranslational modification by identifying each subset of particles and detecting the presence or absence of the first detection reagent; or a combination thereof.

127.-148. (Cancelled).
